(19) United States
(12) Patent Application Publication
(10) Pub. No.: US 2003/0233197 A1
(43) Pub. Date: Dec. 18, 2003

(54) Discrete bayesian analysis of data

(76) Inventors: Carlos E. Padilla, Lexington, MA (US); Valeri I. Karlov, Framingham, MA (US)

Correspondence Address: Stephanie Seidman, Heller Ehrman White & McAuliffe LLP, 4350 La Jolla Village Drive, 7th Floor, San Diego, CA, 92122, US

(21) Appl. No.: 10/394,328

(22) Filed: Mar. 19, 2003

Related U.S. Application Data
(60) Provisional application No. 60/366,441, filed on Mar. 19, 2002.

(51) Int. Cl. G06F 101/14; G06F 17/18; G06F 15/00; G06F 7/00; G06F 9/44; G06N 7/06; G06N 7/02; G06F 17/00; G06N 5/02; G01N 33/50; G01N 33/48; G06F 19/00

(52) U.S. Cl. 702/20; 706/46; 706/52; 706/924; 707/104.1; 702/19; 702/179

(57) ABSTRACT

A probabilistic approximation of a data distribution is provided, wherein uncertain measurements in data are fused together to provide an indication of whether a new data item belongs to a given disease model. The probabilistic approximation is provided in accordance with a Bayesian analysis technique that examines the relationship of probability distributions for observable events x and multiple hypotheses H regarding those events.

### RELATED APPLICATIONS

Benefit of priority is claimed to U.S. Provisional Patent Application Serial No. 60/366,441, filed Mar. 19, 2002 to Padilla et al. entitled “Discrete Bayesian Analysis Of Data”. This application is also related to International PCT application No. (attorney docket no. 24737-1918PC), filed Mar. 19, 2003.

The disclosures of the above-referenced provisional patent application and international PCT application are hereby incorporated herein by reference in their entirety.

### FIELD

Provided herein are methods of mining and analyzing gene expression data to generate clinically relevant information. Also provided are methods for formulating clinically relevant information from sample data.

### BACKGROUND

In the area of disease diagnosis and detection, clinical tests are used to obtain data regarding a patient. The clinical tests yield a large volume of data, including patient symptoms and test results, as well as patient characteristics, such as age, gender, geographic location, and weight. The data may vary depending on the progression of a particular disease and when the clinical tests are conducted on a patient. The amount of clinical test data cumulates as additional tests are performed on an increasing number of patients.

The multitude of clinical test data that is available does not necessarily lead to an improvement in disease diagnosis for a patient. Indeed, the opposite can be true, as the volume of clinical test data and the high dimensionality of such data leads to a large quantity of possible diagnoses that can result from the data. A single patient may have multiple diagnoses that could result from the same data set. Additionally, the data may contain patterns that are not readily apparent or could contain information related to diseases which are not commonly diagnosed, difficult to diagnose, or for which a diagnostic test is not available or does not exist. This can lead to an inefficient use of clinical data wherein the analysis of the data leads to improper diagnoses or to a missed diagnoses due to a failure to spot patterns or connections in the data.

This is also true in the case of other highly multi-dimensional data sets, such as gene expression data. The problems associated with clinical data analysis as described above are compounded when data sets of increasing dimensionality are employed.

In view of the foregoing, it should be apparent that there is a need for a method of mining and analyzing gene expression data in connection with disease diagnosis.

### SUMMARY

In the methods herein, the expression of genes, typically in response to a condition or other perturbation, such as disease, disorder or drug, is assessed to identify patterns of expression. Any method by which the expression of genes can be assessed can be used. For example, gene chips, which contain oligonucleotides representative of all genes or particular subsets thereof, can used. It is understood, however, that any method for assessing expression of a gene can be used. Once patterns of gene expression responsive to conditions or other perturbations are identified, they can be used to predict outcomes of other conditions or perturbations or to identify conditions or perturbations, for diagnosis or for other predictive analyses. Genes assessed include, but are not limited to, genes that are indicative of the propensity to develop diseases that include, but are not limited to diabetes, cardiovascular diseases, cancers, reproductive diseases, gastrointestinal diseases; genes diagnostic of a disease or disorder and genes that are indicative of compound toxicity. Hence the methods herein can be prognostic and/or diagnostic.

Provided herein is a probabilistic approximation of a data distribution, wherein uncertain measurements in data including gene expression data, are fused together to provide an indication of whether a new data item belongs to a given model of clinically relevant information.

In accordance with the methods provided herein, it is possible to handle the more complex situation wherein each patient record has more than one outcome D associated with it. A Markov net probabilistic model is used to model the known (inferred from the training data) probabilities of multiple outcomes. A subset of the set of possible combinations of clinically relevant information is chosen that is mutually exclusive a priori in order to properly formulate the Bayesian inference mechanism.

The methods provided herein make use of the Bayesian relationship for probability distributions for observable events x and multiple hypotheses H regarding those events. In particular, the methods utilize a matrix X of observed gene expression data, wherein each column of the matrix X represents the expression of a different gene and each row of X represents the gene expression data as produced from a single patient or test subject. A column vector D represents a set of outcomes such that each test subject is associated with one outcomes, and each test subject in a row of the X matrix is the same test subject as the corresponding element of the D vector. Thus, the set of H possible outcomes is mutually exclusive. The set of outcomes is selected from among a set H of outcome hypotheses. In a simple example, the set of diagnoses outcomes D may comprise “healthy” and “not healthy”. For a new gene expression data x, the method provided herein produces the probability that a given one of the H hypotheses will be the outcome associated with the gene expression data x, a probability that is written as p(H/x), by utilizing the Bayesian relationship given by

p(H|x)=p(x) * p(x|H)p(H|x)=[p(H)/p(x)] * p(x|H)

wherein p(H) is the a priori probability of the hypothesis H, p(x) is the probability of an outcome, p(x|H) is the conditional probability that specifies the likelihood of obtaining a result x given a hypothesis H. The value p(H|x) is produced despite difficulties that are commonly experienced with conventional techniques for calculating the p(x|H) term.

In one embodiment, the p(x/H) hypothesis-conditional probability density function is approximated by a fusion technique that provides an effective mechanism of decomposition of a high-dimensional space (tens, hundreds, or thousands of genes) still retaining essential statistical dependencies. First, the coarse density estimate is constructed globally using a minimax-type approximation in a form of guaranteeing ellipsoids. Second, the density estimate is corrected locally for each new data point x using the novel discrete patterns of class distributions. The fusion in a very high-dimensional space (thousands of genes) involves additional novel techniques such as a correlation-wave decomposition of the space of genes into essentially correlated subspaces as well as fuzzy clustering techniques based on probabilistic methodology. That is, an approximation of the Bayesian a posteriori distribution is provided. The approximation can advantageously reduce the effect of incomplete or missing data from the data matrix X.

The methods provided herein have application to a variety of data analysis situations, including the use of gene expression microarray data exclusively or in combination with other measurements or data (e.g., clinical tests, for applications such as cell biology (to discover gene function), drug discovery (for new target identification; toxicity studies; drug efficacy), clinical trials (in survivability prediction), medical diagnostics (in disease diagnostics; patient subgroup identification for treatment specialization; disease stage; disease outcome, disease reoccurrence), and systems biology (such as the identification and update of in silico models of “personal molecular states”, as described by Stephen H. Friend and Roland B. Stoughton in Scientific American magazine, February 2002, p. 53).

In another embodiment, a system and method of data diagnosis involves the fusing of uncertain measurements and data with biochemical, biological, and/or biophysical information content for the purposes of predictive model building, hidden pattern recognition, and data mining to predict properties or classifications in applications such as: disease diagnosis, disease stage, disease outcome, disease reoccurrence, toxicity studies, clinical trial outcome prediction and drug efficacy studies. In accordance with the methods provided herein, a detailed probabilistic model for property prediction is derived using relevant data such as can be obtained from gene expression microarrays. The probabilistic model can be used to optimize measurement and data gathering for the application in order to improve relevant property prediction or classification. In this way, the method identifies and takes advantage of cooperative changes in different measurements (e.g., different gene expression patterns) to extract maximum information for prediction. One of the ways to identify cooperative and dependent changes, as well as measurement variability over classes, is through (unsupervised) fuzzy clustering. Fuzzy clustering also can serve as a basis for probabilistic variable reduction for handling high-dimensional measurement spaces. The method can also take into account structural knowledge, such as data trends in time and in the compound/patient/test space, both linear and nonlinear. The method can be employed recursively and can incorporate new information, both quantitative and qualitative, to update the predictive model as more data/measurements become available.

### DESCRIPTION OF DRAWINGS

FIG. 1 depicts geometric illustration of the generalized minimax approach which shows how the fuzzy density estimate (fuzzy due to the non-zero confidential intervals for the covariance matrix) is approximated by a guaranteeing density estimate.

FIG. 2 shows two different examples of decomposing the space of features S into two subspaccs S₁ and S_{L}.

FIG. 3 depicts Geometrical Illustration of the Multiple-Set density FIG. 4 illustrates a general idea of the concept of soft thresholds, which is formalized via a novel way of estimating density locally.

FIG. 5 illustrates the transformation of a local distance space around a new patient, given the global estimates of density.

FIG. 6 shows a geometrical illustration of the neighbor counting patterns for two diagnoses (diagnoses 1 and 2).

FIG. 7 illustrates the transformation of a local distance space around a new patient, given the global estimates of density.

FIG. 8 shows a geometrical illustration of the neighbor counting patterns for two diagnoses (diagnoses 1 and 2).

FIG. 9 illustrates the mechanism of truncation while pairing correlations.

FIG. 10 illustrates clustering of correlations.

FIG. 11 depicts clustered pair-wise operations.

FIG. 12 depicts pair-wise operations for elements within clustered covariance matrix.

FIG. 13 illustrated the DBA for diagnostics from gene expression data.

FIG. 14 shows realistic robost clustering.

FIG. 15 shows hierarchy of robost clusters.

FIG. 16 shows ranking of genes in realistic and optimistic approach.

FIG. 17 shows ranking of some predictive genes in the correlation method and the DBA FIG. 18 shows comparison of DBA performance with the performance of the Gene-Prognosis correlation method in terms of specificity and sensitivity in discriminating the good and poor prognoses.

FIG. 19 shows some predictive genes of the DBA selected in Monte-Carlo runs.

### DETAILED DESCRIPTION

A. Definitions

As used herein, “a discrete Bayesian analysis” refers to an analysis that uses a Bayes conditional probability formula as the framework for an estimation methodology. The methodology combines (1) a nonlinear update step in which new gene expression data is convolved with the a priori probability of a discretized state vector of a possible outcome to generate an a posteriori probability; and (2) a prediction step wherein the computer 110 captures trends in the gene expression data, such as using a Markov chain model of the discretized state or measurements. Such analysis has been adapted herein for processing gene expression data.

As used herein, probabilistic model refers to a model indicative of a probable classification of data, such as gene expression data, to predict outcome, such as disease diagnosis, disease outcome, compound toxicity and drug efficacy.

As used herein, trends refer to patterns of gene expression.

As used herein, dependencies among data refers to relationship between patterns of gene expressions and prediction of clinically relevant information.

As used herein, probability distribution function of stochastic variables refers to a mathematical function that represents probabilities associated with each of the possible outcome, such as disease diagnosis, disease outcome, compound toxicity and drug efficacy based on random variables, such as the gene expression patterns.

As used herein, conditional probability refers to the probability of a particular outcome, such as disease diagnosis, compound toxicity, disease outcome or drug efficacy, given one or more events or variables such as patterns of gene expression.

As used herein, probability density function refers to a mathematical function that represents distribution of possible outcomes from gene expression data.

As used herein, clinically relevant information refers to information obtained from gene expression data such as compound toxicity in general patient population and in specific patients; toxicity of a drug or drug candidate when used in combination of another drug or drug candidate, disease diagnosis (e.g. diagnosis of inapparent diseases, including those for which no pre-symptomatic diagnostic is available, or those for which pre-symptomatic diagnostics are of poor accuracy, and those for which clinical diagnosis based on symptomatic evidence is difficult or impossible); disease stage (e.g., end-stage, pre-symptomatic, chronic, terminal, virulant, advanced, etc.); disease outcome (e.g., effectiveness of therapy; selection of therapy); drug or treatment protocol efficacy (e.g., efficacy in the general patient population or in a specific patient or patient sub-population; drug resistance) risk of disease, and survivability in a disease or in clinical trial (e.g., prediction of the outcome of clinical trials; selection of patient populations for clinical trials).

As used herein, diagnosis refers to a finding that a disease condition is present or absent or is likely present or absent. Hence a finding of health is also considered a diagnosis herein. Thus, as used herein, diagnosis refers to a predictive process in which the presence, absence, severity or course of treatment of a disease, disorder or other medical condition is assessed. For purposes herein, diagnosis also includes predictive processes for determining the outcome resulting from a treatment.

As used herein, subject includes any organism, typically a mammal, such as a human, for whom diagnosis is contemplated. Subjects are also referred to as patients.

As used herein, gene expression refers to the expression of genes as detected by mRNA expressed or products produced from mRNA, such as encoded proteins or cDNA.

As used herein, gene expression data refers to data obtained by any analytical method in which gene products, such as mRNA, proteins or other products of mRNA are detected or assessed. For example, if a gene chip is employed, the chip can contain oligonucleotides that are representative of particular genes. If hybrids between mRNA (or cDNA produced therefrom) are produced at particular loci, the identity of expressed genes can be determined.

As used herein, a perturbuation refers to any input (i.e. exposure of an organism or cell or tissue or organ thereof) or condition that results in an response, as assessed by gene expression. Gene expression includes genes of an affected subject, such as a animal or plant, and also foreign genes such as viral genes in an infected subject. Perturbations include any internal or external change in the environment that results in an altered response compared to in the absence of the change. Thus, for example, as used herein, a perturbation with reference to cells refers to anything intra- or extra-cellular that alters gene expression or alters a cellular response. A perturbation with reference to an organism, such as a mammal, refers to anything, such as drug or a disease that results in an altered response or a response. Such responses can be assessed by detecting changes in gene expression in a particular, cell, tissue or organ, such as tumor tissue or tumor cells or diseased tissue. Perturbations, in one embodiment include, drugs, such as small effector molecules, including, for example, small organics, antisense, RNA and DNA, changes in intra or extracellular ion concentrations, such as changes in pH, Ca, Mg, Na and other ions, changes in temperature, pressure and concentration of any extracellular or intracellular component. The response assess is toxicity. In other embodiments, perturbations refer to disease conditions, such as cancers, reproductive diseases, inflammatory diseases, cardiovascular diseases, and the response assessed is gene expression that is indicative or peculiar to the disease. Any such change or effector or condition is collectively referred to as a perturbations.

As used herein, inapparent diseases (used interchangeably with unapparent diseases) include diseases that are not readily diagnosed, are difficult to diagnose, diseases in asymptomatic subjects or subjects experiencing non-specific symptoms that do not suggest a particular diagnosis or suggest a plurality of diagnoses. They include diseases, such as Alzheimer's disease, Chron's disease, for which a diagnostic test is not available or does not exist. Diseases for which the methods herein are particularly suitable are those that present with symptoms not uniquely indicative of any diagnosis or that are present in apparently healthy subject. To perform the methods herein, a variety data from a subject presenting with such symptoms or healthy are performed. The methods herein permit the clinician to ferret out conditions, diseases or disorder that a subject has and/or is a risk of developing.

As used herein, sensitivity refers to the ability of a search method to locate as many members of data points, such as predictive genes in gene expression dataset, as possible.

As used herein, specificity refers to the ability of a search method to locate members of one family, such as predictive genes responsible for a particular outcome, in a data set, such as gene expression dataset, as possible.

As used herein, a collection contains two, generally three, or more elements.

As used herein, an array refers to a collection of elements, such as oligonucleotides, including probes, primers and/or target nucleic acid molecules or fragments thereof, containing three or more members. An addressable array is one in which the members of the array are identifiable, typically by position on a solid phase support or by virtue of an identifiable or detectable label, such as by color, fluorescence, electronic signal (i.e. RF, microwave or other frequency that does not substantially alter the interaction of the molecules or biological particles), bar code or other symbology, chemical or other such label. Hence, in general the members of the array are immobilized to discrete identifiable loci on the surface of a solid phase or directly or indirectly linked to or otherwise associated with the identifiable label, such as affixed to a microsphere or other particulate support (herein referred to as beads) and suspended in solution or spread out on a surface. Thus, for example, positionally addressable arrays can be arrayed on a substrate, such as glass, including microscope slides, paper, nylon or any other type of membrane, filter, chip, glass slide, or any other suitable solid support. If needed the substrate surface is functionalized, derivatized or otherwise rendered capable of binding to a binding partner. In some instances, those of skill in the art refer to microarrays. A microarray is a positionally addressable array, such as an array on a solid support, in which the loci of the array are at high density. For example, a typical array formed on a surface the size of a standard 96 well microtiter plate with 96 loci, 384, or 1536 are not microarrays. Arrays at higher densities, such as greater than 2000, 3000, 4000 and more loci per plate are considered microarrays.

As used herein, a substrate (also referred to as a matrix support, a matrix, an insoluble support, a support or a solid support) refers to any solid or semisolid or insoluble support to which a molecule of interest, typically a biological molecule, organic molecule or biospecific ligand is linked or contacted. A substrate or support refers to any insoluble material or matrix that is used either directly or following suitable derivatization, as a solid support for chemical synthesis, assays and other such processes. Substrates contemplated herein include, for example, silicon substrates or siliconized substrates that are optionally derivatized on the surface intended for linkage of oligonucleotides.

Such materials include any materials that are used as affinity matrices or supports for chemical and biological molecule syntheses and analyses, such as, but are not limited to: polystyrene, polycarbonate, polypropylene, nylon, glass, dextran, chitin, sand, pumice, polytetrafluoroethylene, agarose, polysaccharides, dendrimers, buckyballs, polyacrylamide, Kieselguhr-polyacrylamide non-covalent composite, polystyrene-polyacrylamide covalent composite, polystyrene-PEG (polyethyleneglycol) composite, silicon, rubber, and other materials used as supports for solid phase syntheses, affinity separations and purifications, hybridization reactions, immunoassays and other such applications.

Thus, a substrate, support or matrix refers to any solid or semisolid or insoluble support on which the molecule of interest, such as an oligonucleotide, is linked or contacted. Typically a matrix is a substrate material having a rigid or semi-rigid surface. In many embodiments, at least one surface of the substrate is substantially flat or is a well, although in some embodiments it can be desirable to physically separate synthesis regions for different polymers with, for example, wells, raised regions, etched trenches, or other such topology.

The substrate, support or matrix herein can be particulate or can be in the form of a continuous surface, such as a microtiter dish or well, a glass slide, a silicon chip, a nitrocellulose sheet, nylon mesh, or other such materials. When particulate, typically the particles have at least one dimension in the 5-10 mm range or smaller. Such particles, referred collectively herein as “beads”, are often, but not necessarily, spherical. Such reference, however, does not constrain the geometry of the matrix, which can be any shape, including random shapes, needles, fibers, and elongated. Roughly spherical “beads”, particularly microspheres that can be used in the liquid phase, are also contemplated. The “beads” can include additional components, such as magnetic or paramagnetic particles (see, e.g., Dyna beads (Dynal, Oslo, Norway)) for separation using magnets, as long as the additional components do not interfere with the methods and analyses herein. For the collections of cells, the substrate should be selected so that it is addressable (i.e., identifiable) and such that the cells are linked, absorbed, adsorbed or otherwise retained thereon.

As used herein, matrix or support particles refers to matrix materials that are in the form of discrete particles. The particles have any shape and dimensions, but typically have at least one dimension that is 100 mm or less, 50 mm or less, 10 mm or less, 1 mm or less, 100 μm or less, 50 μm or less and typically have a size that is 100 mm³ or less, 50 mm³ or less, 10 mm³ or less, and 1 nm³ or less, 100 μm³ or less and can be order of cubic microns. Such particles are collectively called “beads.”

As used herein, high density arrays refer to arrays that contain 384 or more, including 1536 or more or any multiple of 96 or other selected base, loci per support, which is typically about the size of a standard 96 well microtiter plate. Each such array is typically, although not necessarily, standardized to be the size of a 96 well microtiter plate. It is understood that other numbers of loci, such as 10, 100, 200, 300, 400, 500, 10ⁿ, wherein n is any number from 0 and up to 10 or more. Ninety-six is merely an exemplary number. For addressable collections that are homogeneous (i.e. not affixed to a solid support), the numbers of members are generally greater. Such collections can be labeled chemically, electronically (such as with radio-frequency, microwave or other detectable electromagnetic frequency that does not substantially interfere with a selected assay or biological interaction).

As used herein, a gene chip, also called a genome chip and a microarray, refers to high density oligonucleotide-based arrays. Such chips typically refer to arrays of oligonucleotides designed for monitoring an entire genome, but can be designed to monitor a subset thereof. Gene chips contain arrayed polynucleotide chains (oligonucleotides of DNA or RNA or nucleic acid analogs or combinations thereof) that are single-stranded, or at least partially or completely single-stranded prior to hybridization. The oligonucleotides are designed to specifically and generally uniquely hybridize to particular polynucleotides in a population, whereby by virtue of formation of a hybrid the presence of a polynucleotide in a population can be identified. Gene chips are commercially available or can be prepared. Exemplary microarrays include the Affymetrix GeneChip® arrays. Such arrays are typically fabricated by high speed robotics on glass, nylon or other suitable substrate, and include a plurality of probes (oligonucleotides) of known identity defined by their address in (or on) the array (an addressable locus). The oligonucleotides are used to determine complementary binding and to thereby provide parallel gene expression and gene discovery in a sample containing target nucleic acid molecules. Thus, as used herein, a gene chip refers to an addressable array, typically a two-dimensional array, that includes plurality of oligonucleotides associate with addressable loci “addresses”, such as on a surface of a microtiter plate or other solid support.

As used herein, a plurality of genes includes at least two, five, 10, 25, 50, 100, 250, 500, 1000, 2,500, 5,000, 10,000, 100,000, 1,000,000 or more genes. A plurality of genes can include complete or partial genomes of an organism or even a plurality thereof. Selecting the organism type determines the genome from among which the gene regulatory regions are selected. Exemplary organisms for gene screening include animals, such as mammals, including human and rodent, such as mouse, insects, yeast, bacteria, parasites, and plants.

B. Gene Chips For Gene Expression Analyses

Addressable collections of oligonucleotides are used to identify and optionally quantify or determine relative amounts of transcripts expressed. The gene expression data thus obtained is used in the methods provided herein to predict clinically relevant information, including, but not limited to, compound toxicity, disease diagnosis, disease stage, disease outcome, drug efficacy, disease reoccurrence, drug side effects, and survivability in clinical trials.

For purposes herein, addressable collections are exemplified by gene chips, which are arrays of oligonucleotides generally linked to a selected solid support, such as a silicon chip or other inert or derivatized surface. Other addressable collections, such as chemically or electronically labeled oligonucleotides also can be used.

Oligonucleotides can be of any length but typically range in size from a few monomeric units, such as three (3) to four (4), to several tens of monomeric units. The length of the oligonucleotide depends upon the system under study; generally oligonucleotides are selected of a complexity that will hybridize to a transcript from one gene only. For example, for the human genome, such length is about 14 to 16 nucleotide bases. If a genome or subset thereof of lower complexity is selected, or if unique hybridization is not desired, shorter oligonucleotides can be used. Exemplary oligonucleotide lengths are from about 5-15 base pairs, 15-25 base pairs, 25-50 base pairs, 75 to 100 base pairs, 100-250 base pairs or longer. An oligonucleotide can be a synthetic oligomer, a full-length cDNA molecule, a less-than full length cDNA, or a subsequence of a gene, optionally including introns.

Gene chip arrays can contain as few as about 25, 50, 100, 250, 500 or 1000 oligonucleotides that are different in one or more nucleotides or 2500, 5000, 10,000, 20,000, 30,000, 40,000, 50,000, 75,000, 100,000, 250,000, 500,000, 1,000,000 or more oligonucleotides that are different in one or more nucleotides. The greater the number of oligonucleotides on the array representing different gene sequences, the more gene expression data can be identified. Thus, in one embodiment, oligonucleotides that hybridize to all or almost all genes in an organism's genome are used. Such comprehensiveness is not required in order to practice the methods herein. In certain embodiments, oligonucleotides that hybridize only to a gene or genes of interest are used (i.e., in the diagnosis of inapparent diseases). The number of oligonucleotides is a function of the system under study, the desired specificity and the number of responding genes desired. Accordingly, oligonucleotide arrays in which all or a subset of the oligonucleotides represent partial or incomplete genomes can be used, for example 0.1-1%, 1-10%, 10-20%, 20-30%, 30-40%, 50-60%, 60-75%, or 75-85%, or more (e.g., 90% or 95%).

Gene chip arrays can have any oligonucleotide density; the greater the density the greater the number of oligonucleotides that can be screened on a given chip size. Density can be as few as 1-10, such as 1, 2, 4, 5, 6, 8 and 10 oligonucleotides per cm². Density can be as many as 10-100, such as 10-15, 15-20, 20-30, 30-40, 40-50, 50-60, 60-70, 70-80 and 90-100, oligonucleotides per Cm² or more. Greater density arrays can afford economies of scale. High density chips are commercially avaiable (i.e. from Affymetrix).

The substrate to which the oligonucleotides are attached include any impermeable or semi-permeable, rigid or semi-rigid, substance substantially inert so as not to interfere with the use of the chip in hybridization reactions. The substrate can be a contiguous two-dimensional surface or can be perforated, for example. Exemplary substrates compatible with hybridization reactions include, but are not limited to, inorganics, natural polymers, and synthetic polymers. These include, for example: cellulose; nitrocellulose; glass; silica gels; coated and derivatized glass; plastics, such as polypropylene, polystyrene, polystyrene cross-linked with divinylbenzene or other such cross-linking agent (see, e.g., Merrifield (1964) Biochenistry 3:1385-1390); polyacrylamides, latex gels, dextran, rubber, silicon, natural sponges, and many others. The substrate matrices are typically insoluble substrates that are solid, porous, deformable, or hard, and have any required structure and geometry, including, but not limited to: beads, pellets, disks, capillaries, hollow fibers, needles, solid fibers, random shapes, thin films and membranes.

For example, in order to rapidly identify a gene whose expression is increased or decreased, each oligonucleotide or a subset of the oligonucleotides of the addressable collection, such as an array on a solid support, can represent a known gene or a gene polymorphism, mutant or truncated or deleted form of a gene or combinations thereof. Transcripts or nucleic acid derived from transcripts, such as RNA or CDNA derived from the RNA, of a cell subjected to a treatment, such as contacting with a test substance or other signal, to the oligonucleotides are hybridized to the gene chip.

In addition the amount of RNA from a cell or nucleic acid derived from RNA of a cell that hybridizes to oligonucleotides of the array can reflect the level of the mRNA transcript in the cell. By labeling the RNA from a cell or nucleic acid derived from RNA, and comparing the intensity of the signal given by the label following hybridization to oligonucleotides of the array, relative or absolute amounts of gene transcript are quantified. Any differences in transcript levels in the presence and absence of the test perturbation are revealed.

Hybridizing transcripts also identify which, if any among the plurality of genes exhibits is increased, such as two- or three-fold or more or decreased, such as six-fold or more, transcript levels in the presence of the test perturbation, such as a substance or stimulus, in comparison to the absence of the test substance or stimulus.

Exemplary conditions for gene chip hybridization include low stringency, in 6X SSPE-T at 37° C. (0.005% Triton X-100) hybridization followed by washes at a higher stringency (e.g., 1 X SSPE-T at 37° C.) to reduce mismatched hybrids. Washes can be performed at increasing stringency (e.g., as low as 0.25 X SSPE-T at 37° C. to 50° C.) until a desired level of specificity is obtained. Hybridization specificity can be evaluated by comparison of hybridization to the test probes with hybridization to the various controls that can be present (e.g., expression level control, normalization control and mismatch controls).

Additional examples of hybridization conditions useful for gene chip and traditional nucleic acid hybridization (e.g., northerns and southern blots) are, for moderately stringent hybridization conditions: 2X SSC/0.1% SDS at about 37° C. or 42° C. (hybridization); 0.5X SSC/0.1% SDS at about room temperature (low stringency wash); 0.5X SSC/0.I% SDS at about 42° C. (moderate stringency wash); for moderately-high stringency hybridization conditions: 2X SSC/0.1% SDS at about 37° C. or 42° C. (hybridization); 0.5X SSC/0.1% SDS at about room temperature (low stringency wash); 0.5X SSC/0.1% SDS at about 42° C. (moderate stringency wash); and 0.1 X SSC/0.1% SDS at about 52° C. (moderately-high stringency wash); for high stringency hybridization conditions: 2X SSC/0.1% SDS at about 37° C. or 42° C. (hybridization); 0.5X SSC/0.1% SDS at about room temperature (low stringency wash); 0.5X SSC/0.1% SDS at about 42° C. (moderate stringency wash); and 0.1X SSC/0.1% SDS at about 65° C. (high stringency wash).

Hybridization signals can vary in strength according to hybridization efficiency, the amount of label on the nucleic acid and the amount of the particular nucleic acid in the sample. Typically nucleic acids present at very low levels (e.g., <1 pM) will show a very weak signal. A threshold intensity can be selected below which a signal is not counted as being essentially indistinguishable from background. In any case, it is the difference in gene expression (test substance or stimulus, treated vs. untreated) that determines the genes for subsequent selection of their regulatory region. Thus, extremely low levels of detection sensitivity are not required in order to practice methods provided herein.

Detecting nucleic acids hybridized to oligonucleotides of the array depends on the nature of the detectable label. Thus, for example, where a calorimetric label is used, the label can be visualized. Where a radioactive labeled nucleic acid is used, the radiation can be detected (e.g with photographic film or a solid state counter). For nucleic acids labeled with a fluorescent label, detection of the label on the oligonucleotide array is typically accomplished with a fluorescent microscope. The hybridized array is excited with a light source at the appropriate excitation wavelength and the resulting fluorescence emission is detected which reflects the quantity of hybridized transcript. In this particular example, quantitation is facilitated by the use of a fluorescence microscope which can be equipped with an automated stage for automatic scanning of the hybridized array. Thus, in the simplest form of gene expression analysis using an oligonucleotide array, quantitation of gene transcripts is determined by measuring and comparing the intensity of the label (e.g., fluorescence) at each oligonucleotide position on the array following hybridization of treated and hybridization of untreated samples.

The use of two-color fluorescence labeling and detection to measure changes in gene expression can be used (see, e.g., Shena et al. (1995) Science 270:467). Simultaneously analyzing cDNA labeled with two different labels (e.g., fluorophores) provides a direct and internally controlled comparison of the mRNA levels corresponding to each arrayed oligonucleotide; variations from minor differences in experimental conditions, such as hybridization conditions, do not affect the analyses.

1) Oligonucleotide Controls

Gene chip arrays can include one or more oligonucleotides for mismatch control, expression level control or for normalization control. For example, each oligonucleotide of the array that represents a known gene, that is, it specifically hybridizes to a gene transcript or nucleic acid produced from a transcript, can have a mismatch control oligonucleotide. The mismatch can include one or more mismatched bases. The mismatch(s) can be located at or near the center of the probe such that the mismatch is most likely to destabilize the duplex with the target sequence under hybridization conditions, but can be located anywhere, for example, a terminal mismatch. The mismatch control typically has a corresponding test probe that is perfectly complementary to the same particular target sequence.

Mismatches are selected such that under appropriate hybridization conditions the test or control oligonucleotide hybridizes with its target sequence, but the mismatch oligonucleotide does not. Mismatch oligonucleotides therefore indicate whether hybridization is specific or not. For example, if the target gene is present the perfect match oligonucleotide should be consistently brighter than the mismatch oligonucleotide.

When mismatch controls are present, the quantifying step can include calculating the difference in hybridization signal intensity between each of the oligonucleotides and its corresponding mismatch control oligonucleotide. The quantifying can further include calculating the average difference in hybridization signal intensity between each of the oligonucleotides and its corresponding mismatch control oligonucleotide for each gene.

Expression level controls are, for example, oligonucleotides that hybridize to constitutively expressed genes. Expression level controls are typically designed to control for cell health. Covariance of an expression level control with the expression of a target gene indicates whether measured changes in expression level of a gene is due to changes in transcription rate of that gene or to general variations in health of the cell. For example, when a cell is in poor health or lacking a critical metabolite the expression levels of an active target gene and a constitutively expressed gene are expected to decrease. Thus, where the expression levels of an expression level control and the target gene appear to decrease or to increase, the change can be attributed to changes in the metabolic activity of the cell, not to differential expression of the target gene. Virtually any constitutively expressed gene is a suitable target for expression level controls. Typically expression level control genes are “housekeeping genes” including, but not limited to β-actin gene, transferrin receptor and GAPDH.

Normalization controls are often unnecessary for quantitation of a hybridization signal where optimal oligonucleotides that hybridize to particular genes have already been identified. Thus, the hybridization signal produced by an optimal oligonucleotide provides an accurate measure of the concentration of hybridized nucleic acid.

Nevertheless, relative differences in gene expression can be detected without the use of such control oligonucleotides. Therefore, the inclusion of control oligonucleotides is optional.

2) Synthesis of Gene Chips

The oligonucleotides can be synthesized directly on the array by sequentially adding nucleotides to a particular position on the array until the desired oligonucleotide sequence or length is achieved. Alternatively, the oligonucleotides can first be synthesized and then attached on the array. In either case, the sequence and position (i.e., address) of all or a subset of the oligonucleotides on the array will typically be known. The array produced can be redundant with several oligonucleotide molecules representing a particular gene.

Gene chip arrays containing thousands of oligonucleotides complementary to gene sequences, at defined locations on a substrate are known (see, e.g., International PCT application No. WO 90/15070) and can be made by a variety of techniques known in the art including photolithography (see, e.g., Fodor et al. (1991) Science 251:767; Pease et al. (1994) Proc. Natl. Acad. Sci. U.S.A. 91:5022; Lockhartet al.(1996) Nature Biotech 14:1675; and U.S. Pat. Nos. 5,578,832; 5,556,752; and 5,510,270).

A variety of methods are known. For example methods for rapid synthesis and deposition of defined oligonucleotides are also known (see, e.g., Blanchard et al. (1996) Biosensors & Bioelectronics 11:6876); as are light-directed chemical coupling, and mechanically directed coupling methods (see, e.g., U.S. Pat. No. 5,143,854 and International PCT application Nos. WO 92/10092 and WO 93/09668, which describe methods for forming vast arrays of oligonucleotides, peptides and other biomolecules, referred to as VLSIPS™ procedures (see also U.S. Pat. No. 6,040,138)). U.S. Pat. No. 5,677,195 describes forming oligonucleotides or peptides having diverse sequences on a single substrate by delivering various monomers or other reactants to multiple reaction sites on a single substrate where they are reacted in parallel. A series of channels, grooves, or spots are formed on a substrate and reagents are selectively flowed through or deposited in the channels, grooves, or spots, forming the array on the substrate. The aforementioned techniques describe synthesis of oligonucleotides directly on the surface of the array, such as a derivatized glass slide. Arrays also can be made by first synthesizing the oligonucleotide and then attaching it to the surface of the substrate e.g., using N-phosphonate or phosphoramidite chemistries (see, e.g., Froehler et al. (1986) Nucleic Acid Res 14:5399; and McBride et al. (1983) Tetrahedron Lett. 24:245). Any type of array, for example, dot blots on a nylon hybridization membrane (see, e.g., Sambrook et al. (1989) Molecular Cloning: A Laboratory Manual (2nd Ed.), Vol. 1-3, Cold Spring Harbor Laboratory, Cold Spring Harbor, N.Y.) can be used.

3) Gene Chip Signal Detection

As discussed, fluorescence emission of transcripts hybridized to oligonucleotides of an array can be detected by scanning confocal laser microscopy. Using the excitation line appropriate for the fluorophore, or for two fluorophores if used, will produce an emission signal whose intensity correlates with the amount of hybridized transcript. Alternatively, a laser that allows simultaneous specimen illumination at wavelengths specific to the two fluorophores and emissions from the two fluorophores can be used for simultaneously analyzing both (see, e.g., Schena et al. (1996) Genome Research 6:639).

In any case, hybridized arrays can be scanned with a laser fluorescent scanner with a computer controlled X-Y stage and a microscope objective. Sequential excitation of the two fluorophores is achieved with a multi-line, mixed gas laser and the emitted light is split by wavelength and detected with two photomultiplier tubes. Alternatively, other fiber-optic bundles (see, e.g., Ferguson et al. (1996) Nature Biotech. 14:1681) can be used to monitor mRNA levels simultaneously. For any particular hybridization site on the array, a ratio of the emission of the two fluorophores can be calculated. The ratio is independent of the absolute expression level of the gene, but is useful for identifying responder genes whose expression is significantly increased or decreased in response to a perturbation, such as a test substance or stimulus.

C. Exemplary Alternatives To Gene Chip For Expression Analyses

1) Target Arrays

As an alternative, for example, nucleic acid can be linked to a solid support, and collections of probes or oligonucleotides of known sequences hybridized thereto. The probes or oligonucleotides can be uniquely labeled, such as by chemical or electronic labeling or by linkage to a detectable tag, such as a colored bead. The expressed genes from cells exposed to a test perturbation are compared to those from a control that is not exposed to the perturbation. Those that are differentially expressed are identified.

2) Other Non-Gene Chip Methods For Detecting Changes In Gene Expression

In addition to using gene chips to detect changes in gene expression, changes in gene expression also can be detected by other methods known in the art. For example, differentially expressed genes can be identified by probe hybridization to filters (Palazzolo et al. (1989) Neuron 3:527; Tavtigian et al. (1994) Mol Biol Cell 5:375). Phage and plasmid DNA libraries, such as cDNA libraries, plated at high density on duplicate filters are screened independently with cDNA prepared from treated or untreated cells. The signal intensities of the various individual clones are compared between the two filter sets to determine which clones hybridize preferentially to cDNA obtained from cells treated with a test substance or stimulus in comparison to untreated cells. The clones are isolated and the genes they encode are identified using well established molecular biological techniques.

Another alternative involves the screening of CDNA libraries following subtracting mRNA populations from untreated and cells treated with a test substance or stimulus (see, e.g., Hedrick et al. (1984) Nature 308:149). The method is closely related to differential hybridization described above, but the CDNA library is prepared to favor clones from one mRNA sample over another. The subtracted library generated is depleted for sequences that are shared between the two sources of mRNA, and enriched for those that are present in either treated or untreated samples. Clones from the subtracted library can be characterized directly. Alternatively, they can be screened by a subtracted CDNA probe, or on duplicate filters using two different probes as above.

Another alternative uses differential display of mRNA (see, e.g., Liang et al. (1995) Methods Enzymol 254:304). PCR primers are used to amplify sequences from two mRNA samples by reverse transcription, followed by PCR. The products of these amplification reactions are run side by side, i.e., pairs of lanes contain the same primers but mRNA samples obtained from treated and untreated cells on DNA sequencing gels. Differences in the extent of amplification can be detected by any suitable method, including by eye. Bands that appear to be differentially amplified between the two samples can be excised from the gel and characterized. If the collection of primers is large enough it is possible to identify numerous gene differentially amplified in treated versus untreated cell samples.

Another alternative designated Representational Difference Analysis (RDA) of nucleic acid populations from different samples (see, e.g., Lisitsyn et al. (1995) Methods Enzymol. 254:304) can be used. RDA uses PCR to amplify fragments that are not shared between two samples. A hybridization step is followed by restriction digests to remove fragments that are shared from participation as templates in amplification. An amplification step allows retrieval of fragments that are present in higher amounts in one sample compared to the other (i.e., treated vs. untreated cells).

3) Detection of Proteins to Assess Gene Expression

Changes in gene expression also can be detected by changes in the levels of proteins expressed. Any method known to those of skill in the art for assessing protein expression and relative expression, such as antibody arrays that are specific for particular proteins and two-dimensional gel analyses, can be employed. Protein levels can be detected, for example, by enzyme linked immunosorbent assays (ELISAs), immunoprecipitations, immunofluorescence, enzyme immunoassay (EIA), radioimmunoassay (RIA), and Western blot analysis.

An array of antibodies can be used to detect changes in the level of proteins. Biosensors that bind to large numbers of proteins and allow quantitation of protein amounts in a sample (see, e.g., U.S. Pat. No. 5,567,301, which describes a biosensor that includes a substrate material, such as a silicon chip, with antibody immobilized thereon, and an impedance detector for measuring impedance of the antibody) can be employed. Antigen-antibody binding is measured by measuring the impedance of the antigen bound antibody in comparison to unbound antibody.

A biosensor array that binds to proteins are used to detect changes in protein levels in response to a perturbation, such as a test substance or stimulus. For example, U.S. Pat. No. 6,123,819 describes a protein sensor array capable of distinguishing between different molecular structures in a mixture. The device includes a substrate on which nanoscale binding sites in the form of multiple electrode clusters are fabricated in which each binding site includes nanometer scale points extending above the surface of a substrate. These points provide a three-dimensional electro-chemical binding profile which mimics a chemical binding site and has selective affinity for a complementary binding site on a target molecule or for the target molecule itself.

D. Methods

The methods provided herein are applied to the gene expression data obtained as described above in order to predict clinically relevant information. Such clinically relevant information includes, but is not limited to, compound toxicity (e.g., toxicity of a drug candidate) both in the general patient population and in specific patients based on gene expression data; toxicity of a drug or drug candidate when used in combination with another drug or drug candidate (i.e., drug interactions)); disease diagnosis (e.g., diagnosis of inapparent diseases, including those for which no pre-symptomatic diagnostic is available, or those for which pre-symptomatic diagnostics are of poor accuracy, and those for which clinical diagnosis based on symptomatic evidence is difficult or impossible); disease stage (e.g., end-stage, pre-symptomatic, chronic, terminal, virulant, advanced, etc.); disease outcome (e.g., effectiveness of therapy; selection of therapy); drug or treatment protocol efficacy (e.g., efficacy in the general patient population or in a specific patient or patient sub-population; drug resistance) risk of disease, and survivability in a disease or in clinical trial (e.g., prediction of the outcome of clinical trials; selection of patient populations for clinical trials).

Diseases for which the methods provided herein may be used to determine disease outcome, disease stage, disease diagnosis and/or survivability in clinical trials and/or risk of developing a particular disease or condition include any disease for which gene expression data provides a clinically useful information. Such diseases include cancer, including but not limited to ovarian, breast, pancreatic, prostate, brain, lung and colon cancer; solid tumors, melanoma, cardiovascular disease, including but not limited to hypertension, pulmonary hypertension, and congestive heart failure; diabetes; HIV/AIDS; hepatitis, including hepatitis A, B and C; thyroid disease, neurodegenerative disorders, reproductive disorders, cardiovascular disorders, autoimmune disorders, inflammatory disorders, cancers, bacterial and viral infections, diabetes, arthritis and endocrine disorders. Other diseases include, but are not limited to, lupus, rheumatoid arthritis, endometriosis, multiple sclerosis, stroke, Alzheimer's disease, Parkinson's diseases, Huntington's disease, Prion diseases, amyotrophic lateral sclerosis (ALS), ischaemias, atherosclerosis, risk of myocardial infarction, hypertension, pulmonary hypertension, congestive heart failure, thromboses, diabetes mellitus types I or II, disorders of lipid metabolism; and any other disease or disorder for which gene expression data can be used in the methods provided herein to predict clinically relevant information.

1. Discrete Bayesian Analysis

In accordance with the methods provided herein, a probabilistic prediction model is used for data analysis for gene expression data. The probabilistic prediction model permits data analysis to treat gene expression microarray measurements explicitly as realizations of a stoFchastic variable. This recognizes that observations exhibit significant variability, and accordingly treats them probabilistically. The probabilistic prediction also involves techniques that:

Approximate the Class Probability Density Function

Class: Disease type, stage, toxic response, phenotype;

Variable Space: all genes in a microarray experiment.

Create Discrete Bayesian Classifier

Built-in natural confidence measure: a posteriori probability of belonging to a class;

No premature variable selection: use of sparse matrix techniques and correlation wave approach, decomposition into local and global spaces and fuzzy clustering approaches to allow treatment of high-dimensional space; and

No artificial “distance” metric: probabilistic comparison of density functions provides class discrimination and prediction mechanism.

A computer based data analysis provided herein includes various statistical analyses, such as pattern recognition, that are performed on gene expression data in order to identify general trends and dependencies among the data. The analysis is preferably combined with a visualization of the data wherein the data is plotted in various histograms, distributions, and scatter plots in one or more dimensions. The method thus combines data visualization, data analysis, and data fusion to result in enhanced prediction of outcomes.

Visualization helps to confirm whether there is a relatively high degree of discrimination between records with different classifications in the space of measurements/data and also helps to assess the shapes of distributions in measurements, such as single peak distributions, which are sometimes close to the Gaussian distributions but sometimes have a high degree of asymmetry.

Another advantage of visualization is that it shows whether the tails or fringes of N-dimensional distributions of measurements could be a clue to property prediction. Visualization further assists in confirming significant dependency of statistical distributions on the relevant/characteristic data properties (e.g., patient's age and sex).

As part of analysis and visualization, the operation of fuzzy clustering has been found to be important, especially for applications involving gene expression arrays. This operation helps to identify cooperative patterns of gene expression that yield hidden pattern information on a property of interest, and at the same time provide a basis for dimensionality reduction via variable reduction based on a probabilistic measure. A robust clustering algorithm provides a rigorous statistical treatment of variability and overlaps in the data. As a result of this, it generates a reliability measure for gene assignments to clusters.

Fuzziness in the clusters can be due to the variability of the gene expressions over samples and overlaps in the gene expression data. An important point is that genes show different clustering characteristics for the given samples and conditions. Some genes cluster stably and some genes migrate between clusters. There are particular patterns of “cluster interactions.” These patterns are highly correlated with a hierarchical tree of clusters that results from the robust clustering operation (genes tend to “migrate” between similar clusters).

By exposing and probabilistically handling this information, instead of hiding it through arbitrary threshold decisions, additional flexibility is obtained in the subsequent analysis. For example, it is now possible to investigate the “most” stable genes as markers. Better yet, this information is used by the probabilistic predictive model provided herein to reduce the dimensionality of the variable space in a systematic manner that takes into account the uncertainties in, and correlations within, the gene expression measurements.

In the next operation, the computer uses the measurement data to form a probabilistic model that will assist in forming a property prediction (or class assignment) as in a disease diagnosis for a patient. The model is preferably based upon a predictive analysis, such as the discrete Bayesian analysis (DBA). The DBA uses a Bayes conditional probability formula as the framework for an estimation methodology. The methodology combines (1) a nonlinear update step in which new data is convolved with the a priori probability of a discretized state vector of a possible outcome to generate an a posteriori probability; and (2) a prediction step wherein the computer captures trends in the measurement data, such as using a Markov chain model of the discretized state or measurements.

The model can have increasing levels of sophistication, such as nonlinear, non-Gaussian and uncertain statistics models, or trend models of test level variation with various factors, including, for example, age, sex, and disease progression. The increasing levels of sophistication may be configured to more accurately represent the underlying statistics in the measurement data and so improve the model's effectiveness in predicting properties or classes (e.g., outcomes in both sensitivity and specificity measures).

For example, some measurement data may vary with the age of the test subject. A Markov chain model can capture the statistical trends in the data and propagate the distribution of the data between different age groups. Age groups that are remote to a patient may be given a lesser weight when fused into the diagnostic process. This allows the use of data statistics from a broad age window, which is helpful where statistics are low from a particular age window. The DBA captures the patterns of disease progression, thereby providing a dynamic pattern of changes in measurement data that can serve as a more accurate indicator of a disease.

In addition to the probabilistic model, there is in one embodiment, also developed an acceptance criterion that improves the predictive accuracy (e.g., sensitivity and specificity of a statistical test) by allowing only those predictions for which the a posteriori probabilities of certain possible classes exceed a threshold. The threshold is, in one embodiment, relative to the probabilities of all possible classes and can be adjusted to minimize the likelihood of false predictions. The acceptance criterion can also be used as a basis for generating a tree or dendogram of possible classes for each record. The method automatically indicates if the measurements of each individual record fall into the acceptance group for which the success rate of making the right classification is very high, such as greater than 90 percent. However, even if the acceptance percent is small, such as for unapparent diseases, the selectivity allows for highly accurate diagnostics for a large number of patients.

The probabilistic models are in one embodiment, initiated and supplemented by a visualization and analysis approach, particularly for measurement data for which analytical formulations and physical bases are not available. For example, the evaluation of the probabilistic models can include an automated visualization of distributions of measurements in one through n dimensional space for specified selection criteria, such as, for example, age, gender, and other factors. This allows making the optimal decision on the model for density approximation, such as to maintain the model as Gaussian or to use beta-functions to capture asymmetric effects. Visualization also aids the detection of groupings of highly correlated measurements and the development of a sophisticated density approximation of the multi-dimensional density, which accounts for the probability of the data. The visualization and analysis can also help to identify those combinations of genes that are most highly discriminating for a particular disease, thus allowing for variable reduction that further analysis implementations.

In one embodiment, the one or more statistical screening tests arc developed to screen for one or more unapparent diseases, which are not commonly diagnosed, difficult to diagnose, or for which a diagnostic test is not available or does not exist.

As mentioned, the model is in one embodiment, based on the technique that is herein referred to as the DBA, which is described elsewhere herein and which is based upon the fundamental Bayesian formalism. The DBA provides a framework for handling multiple classes by increasing the likelihood of detecting a correct single class over other candidate classes. In dealing with multiple and mutually exclusive classes, the DBA in one embodiment generates a tree of possible classes for each record using the record's measurements. The values of the record's genetic expression data determine how a tree is detailed. The DBA indicates to which acceptance group each record belongs. For example, for a certain percent of records, the DBA could provide a coarse tree of possible classes, while the tree could be more detailed for another percentage of patients.

A Bayesian nets formalism is used to incorporate into the DBA information on how classes usually combine. The Bayesian nets formalism is a generalization of a Markov chain model with transitional probabilities between possible groupings of classes. Such an a priori model of class groupings is supplemented by multiple classes a posteriori information, as the massive database of the measurements contain records that have multiple classes associated with them. The measurements could be fused with additional (e.g., genetic) information to sharpen the tree of possible classes. That is, the DBA has the ability to improve the predictability of the classes from the measurements by correlating them with the additionally known properties (e.g., genetic) of each individual record.

2. Computation

A more detailed description of the computational techniques utilized in the methods herein is provided below.

1. Introduction

This description presents the main mathematical ideas underlying the DBA (Discrete Bayesian Approach) technique in accordance with the methods provided herein and shows how the DBA can be customized to the diagnostics problem from gene expression data.

The DBA technique is based on the fundamental Bayesian inference mechanism but goes far beyond by offering two important features:

1. New effective robust algorithms to fuse large amount of high-dimensional data; and

2. Unique customization to the physical structure of a particular problem.

Given its advanced mathematical algorithms and a highly customizable methodology, the DBA technique makes it possible to fuse all available statistical and structural information in order to extract maximum knowledge from the experiments.

There are significant differences between the DBA technique for analysis of gene expression data and a “classical Bayesian analysis.” In the classical analysis, usually not more than one data set is considered in order to generate the posterior probabilities of a disease state, effectively the positive predictive value. The problem is then relatively straightforward and an estimate of the class probability density function for the test is usually a normal distribution, which is good enough if there is sufficient data. The DBA implementation here described goes significantly beyond this naive implementation. First, its aim is to “fuse” information from hundreds to thousands of tests, not one or two. The multi-dimensional class probability density function presents a formidable estimation problem. Approximation of a naive implementation of a multi-Gaussian distribution, would result in the covariance matrix which is extremely large (1000's by 1000's) and cause numberless computational bottlenecks. It would be hard to estimate the correlations with any accuracy in the absence of very large amounts of data, and even in this case, a nafve Gaussian approximation would over-guarantee the probabilities. What is needed is a sophisticated approach to density estimation that can work computationally in very high dimensional spaces and that can handle realistic properties of the data, such as sparsity, uncertainty, and correlations. The description of the DBA technique below focuses on these unique, innovative and highly useful features to estimate the conditional class probability density function for the multi-dimensional vector of tests.

2. Mathematical Statement of Diagnostics Problem

The mathematical statement of the conventional diagnostics problem can be formulated as a standard classification problem (supervised learning).

The formulation starts from the availability of two major pieces of information:

1) Matrix of observed tests

$$X = ( \begin{matrix}\underline{{x}_{1}} \\ \underline{{x}_{2}} \\ ⋮ \\ \overline{{x}_{n}}\end{matrix} ) = ( \begin{matrix}{x}_{11} & {x}_{12} & ⋯ & {x}_{1 m} \\ {x}_{21} & {x}_{22} & ⋯ & {x}_{2 m} \\ ⋮ & ⋮ & ⋮ & ⋮ \\ {x}_{n1} & {x}_{n2} & ⋯ & {x}_{n m}\end{matrix} ) \qquad ( 1 )$$

Here the matrix X is of size n×m and its elements are the test values (gene expressions, etc.), n is the number of patients and m is the number of distinct tests (features). Correspondingly, the observation 1×m vector xᵢ is associated with each patient. A realistic practical situation is assumed when not each patient has a complete list of tests (from all m possible).

2) Vector of diagnosis

$$D = ( \begin{matrix}{D}_{1} \\ {D}_{2} \\ ⋮ \\ {D}_{n}\end{matrix} ) \qquad ( 2 )$$

Here the vector D is of size n×1. The diagnoses are assigned by doctors to each patient, and serve as classification labels. It is assumed that the diagnosis Dᵢ (for i-th patient) is defined on a discrete set of hypotheses (classes): H={H₁, H₂, . . . , H_{N}}. In this conventional statement it is assumed that the hypotheses are mutually exclusive and are also correct with the probability 1.

The goal is to use the combined data {X, D} (tests matrix X and diagnoses vector D) as a training set to develop a predictive diagnostics algorithm. A diagnosis D_{new} (from the possible, ones: H₁, H₂, . . . , H_{N}) is assigned to each new patient who has a set of measured tests x_{new}. The assigned diagnosis should be “the best” in the sense of capturing the statistical dependency of the diagnoses D on the tests X in the {X, D} training set. There are different concepts how to interpret “the best”. It is believed that the BEST (Bayesian ESTtimation) offers the best inference mechanism that leads to the evaluation of a posteriori probabilistic measure p(·) over a set of hypotheses H={H₁, H₂, . . . , H_{N}}:

p(H/x_{new})={p(H₁/x_{new}), p(H₂/x_{new}), . . . , p(H_{N}/x_{new})} (3)

In Eq. (3) the probabilities are conditioned on the observation x_{new}.

The probabilistic information of Eq. (3) is used in the decision making process, which is usually based on the rule of maximum a posteriori probability:

$$\hat{H} = {H}_{\hat{k}} , \hat{k} = arg {max}_{k} p ( {H}_{k} \vert {x}_{new} ) k = 1 , … , N \qquad ( 4 )$$

Elaboration of this rule, especially in conjunction with the acceptance criterion, will be presented in elsewhere as a part of the DBA.

It is important to note that this probabilistic interpretation is possible due to the statistical nature of the diagnostics problem and is desirable from a practical point of view since a likelihood of each diagnosis is assessed.

The predictive diagnostics algorithm should work on each patient individually. However, it is important to evaluate statistical criteria that would characterize the overall quality of predictions on a large set of patients. In other words, the statement of the diagnostics problem should include a cross-validation procedure. It entails a splitting of the available data into two subsets: a training set and a control set. For simplicity, notation X−D for a training set is retained and a structurally equivalent control set is denoted as X_{C}−D_{C} (X_{C} of size n_{C}×m and D_{C} of size n_{C}×1). In this case, after training the predictive algorithm on the X−D data, this algorithm is used for diagnostics of the “new” patients from the control set. The predictive algorithm evaluates the “new” diagnoses D_{C} for all “new” patients. For this set the correct (as assumed) diagnoses D_{C} are available. The mismatch between the correct diagnoses (D_{C}) and predicted diagnoses (\hat{D}_{C}) is the subject for analysis in order to evaluate the conventional statistical criteria such as sensitivity and specificity (see Section 3) the new criterion of acceptance (see Section 3) and ultimately predictive values. From a practical point of view, it is useful to perform a large number of random splits of the original data into different training and control sets. This so-called “boot-strapping” procedure or basically Monte-Carlo simulation makes it possible to estimate the distributions and parameters of the primary statistical criteria (sensitivity, specificity, acceptance and predictive values).

2.1 Challenges of Diagnostics Problem

Here the main challenges of the conventional diagnostics problem (Tests-Diagnoses), i.e. mainly computational challenges of the diagnostics problem, are emphasized. These challenges are associated with the key operation of the Bayesian-type algorithm—estimation of the hypothesis-conditional PDF (Probability Density Function) in the space of tests: p(x/Hₖ), k=1, . . . , N. The challenges are the following:

High dimensionality of the space of tests

Non-Gaussian distributions of tests

Uncertain statistics (especially correlations) due to finite samples and sparsity

Significant overlaps in the tests distributions (It should be noted that although some other classification techniques such as NN or SVM do not use a probabilistic interpretation, they still face the challenges listed above. Although they address these challenges in ways different than the probabilistic methods do, they do not have the benefits of the probabilistic methods.)

Provided below is some elaboration on the challenges listed above, which are highly intertwined.

The challenge of high dimensionality (a so-called curse of dimensionality) might be significant even if the number of tests is equal to 5-6. Indeed, even with these dimensions of x it becomes difficult to evaluate and memorize the hypothesis-conditional PDF p(x/Hₖ),k=1, . . . , N, if the latter is non-Gaussian. The situation quickly aggravates with the increase of tests, making a direct non-parametric estimation of density simply infeasible. The parametric density estimation procedures, e.g. based on Gaussian approximations involving the estimates of the mean vector and covariance matrix, significantly alleviate the curse of dimensionality. But, again, if the density is significantly non-Gaussian or if it is difficult to parameterize it by any other functional form (e.g. β-function), the parametric methods become inaccurate.

Uncertainties in statistics are caused by the fact that typically there is a limited number of patients with the specified tests X (finite samples) and, to make matters worse, not each patient has all tests recorded (sparsity). Under these conditions it is difficult to estimate the density p(x/Hₖ), k=1, . . . , N. especially in the high-dimensional space of tests. Correspondingly, the estimated statistics p(x/Hₖ), k=1, . . . , N to be used in the predictive algorithm are uncertain. The most challenging technical difficulty here consists in the fact that the correlations (or more generally, statistical dependencies) become uncertain, which significantly complicates the fusion of those tests. It is a well-known fact that from finite samples it is more difficult to estimate the entire matrix of pair-wise correlations between all tests rather than the diagonal of this matrix (variances of tests). It is even more difficult to estimate higher order momenta, which formalize statistics of groupings of multiple tests. In addition to finite samples, the sparsity in the available data further complicates the density estimation, especially in terms of estimating mutual statistical dependencies between the test values.

The poor estimates of the density \hat{p}(x/Hk), k 1, . . . , N could introduce large errors to the predictive algorithm especially in the case when the densities for each hypothesis are overlapped. These overlaps are typical for gene expression data. The paradox here is the following. On the one hand, it is beneficial to handle the overlapped distributions via the use of probabilistic measure for fusing a large amount of relatively low-discriminative tests. On the other hand, the accurate estimate of density is problematic. It should be also mentioned that in the case of gene expression data the dimension of the feature space is very high (thousands of genes), which creates an additional challenge due to overlaps. Indeed, a practical approach here usually employs data clustering (unsupervised learning) for reducing the dimensionality of the feature space. Overlaps of the data in the feature space complicate the clustering procedure and require coupling of this procedure with the predictive algorithm.

In summary, it is widely recognized that it is a challenging mathematical problem to fuse the realistic data (high-dimensional, non-Gaussian, statistically uncertain due to finite samples and sparsity, and highly-overlapped). To put it in numbers, the real art of the data fusion consists in developing the robust algorithms to achieve the discrimination probability of 0.85-0.99 for a combination of multiple tests with the individual discrimination probabilities of 0.55-0.7.

3. Data Fusion via the DBA Algorithms

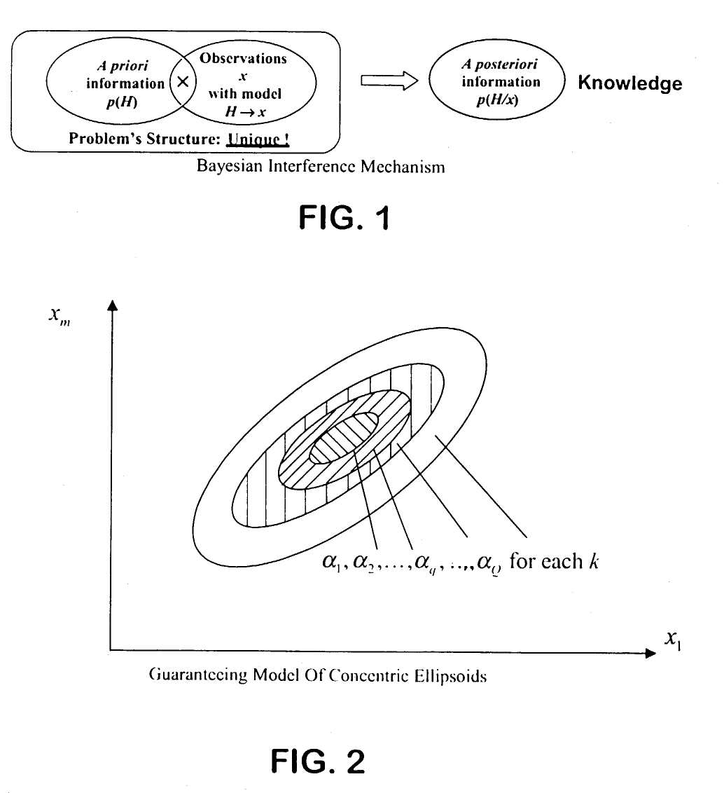

The DBA technology provided herein offers a rigorous statistical treatment of the realistic uncertain data. The DBA technology offers a powerful data fusion framework to extract hidden patterns of diseases in a high-dimensional space of gene expressions data. The DBA technology takes its roots in the classical Bayesian inference mechanism. FIG. 1 provides a graphical interpretation of the Bayesian interference mechanism, as used it in the design of the DBA.

Eq. (5) is the Bayesian formula and it is at the heart of the DBA's computational engine:

$$p ( {H}_{k} / x ) = p ( {H}_{k} ) \cdot \frac{p ( x / {H}_{k} )}{{\sum }_{k = 1 , … , N} p ( {H}_{k} ) p ( x / {H}_{k} )} , k = 1 , … , N \qquad ( 5 )$$

As was described above, H stands for hypotheses (diagnoses), x stands for observed tests (it serves as an input argument), and p(·) is a probabilistic measure. In particular, p(Hₖ), k=1, . . . , N are the a priori probabilities for hypotheses and p(x/Hₖ), k=1, . . . , N are the hypothesis-conditional PDFs, which are represented (in the diagnostics problem) by their estimates. When using Eq. (5) for diagnostics of a new patient who has the vector of tests x_{new}, one just needs to use a substitution x=x_{new}.

The fundamental nature of the Bayesian formula provides a mathematical basis for data fusion. The Bayesian formula provides an advanced mathematical operation (comparing with the arithmetic operations + − ×:) to deal with fuzziness of real world data. This operation involves a probabilistic measure p(·)ε[0,1] for seamless addition (fusion, integration) of different pieces of information, especially in the problems with complex physical structure. From a practical point of view, this operation provides a powerful mechanism for recursively incorporating new information, both quantitative and qualitative, to update the predictive model as more data/measurements become available.

As was mentioned above, the DBA is based on the fundamental Bayesian interference mechanism of Eq. (5), but offers two major types of innovations:

1. New effective robust algorithms to fuse large amount of high-dimensional data.

2. nique customization to the physical structure of a particular problem.

Correspondingly, the first type of innovations addresses the challenges of the conventional diagnostics problem (see Section 2.1), which are mainly mathematical (computational) challenges. The second type of innovations addresses the challenges of the practical diagnostics problem.

To accomplish the first type of innovations, the DBA has important features such as efficient operations in the high-dimensional space of tests and robustness to data variability (including uncertain statistics). These innovations are described in detail in Section 3.1.

To accomplish the second type of innovations, the DBA offers new opportunities to incorporate the structure of a particular problem. This structure includes key factors that differentiate the data under analysis. The DBA has training and prediction modes. In the training mode, the DBA uses two conventional inputs for supervised learning as well as a third unique input through which the problem's structure is formalized. For example, for the medical diagnostics problem, statistical trends in gene expression data with structural data that includes age and combinations of diseases is formalized (using various stochastic models like Markov chains). In the prediction mode for new patients, the trained DBA maps the gene expression data into the a posteriori tree of diagnoses. The information content of this tree sharpens as new gene expression data is added. In this sense, the DBA extracts maximum knowledge and is much less sensitive to problems that arise from data variability. Other general-purpose classification techniques (such as neural nets and support-vector learning machines) lack this ability to be customized to the specific nature of the problem and thus to extract maximum information from the available data, given structural information. For example, the DBA's ability to incorporate the biological information for gene expression data could go as far as development of Bayesian nets for modeling biological pathways and gene regulation processes.

3.1 The DBA for Solving the Conventional Diagnostics Problem (Mathematical Innovations)

The key algorithmic problem in designing the DBA predictive algorithm consists is the estimation of the hypothesis-conditional PDF (Probability Density Function): p(x/Hₖ), k=1, . . . , N. The challenges of this operation were discussed in Section 2.1. In overcoming these challenges the density should be estimated in a form and to an extent, which are sufficient for the development of an accurate prediction (classification) algorithm, in terms of evaluating reliable a posteriori probabilities p(H/x_{new}).

The DBA offers new effective algorithms for density estimation and, thus, opens the way for fusing large high-dimensional datasets. In the following Section these algorithms highlighting the two highly interconnected aspects of the DBA are described: 1) efficient operations in high dimensional space; and, 2) robustness to uncertainties.

3.1.1 Efficient and Robust Operations in the High-Dimensional Space Of Tests

In this Section two different but complementary techniques for operating with high-dimensional data are differentiated. First, Section 3. 1. 1. 1 presents the decomposition techniques tailored for handling tens or hundreds of tests (typical for gene expression data). Second, Section 3.1.1.2 presents clustering techniques tailored for handling very large dimensions with thousands of tests and beyond (typical for gene expression data). It should be noted that clustering should be considered as a technique for reducing the data to a point where the decomposition techniques can be used on the clustered data.

3.1.1.1 Decomposition Techniques

The decomposition techniques are based on the novel idea of global-local estimation of the hypothesis-conditional density p(x/Hₖ), k=1, . . . , N. Correspondingly, the DBA includes a combination of global and local estimates. The estimate is called global when the density is estimated over the entire region of the test values. The estimate is called local if it is associated with a local region in the space of tests.

The state-of-the-art pattern recognition methods use the global and local estimates separately. For example, the Bayesian-Gaussian parametric method (see e.g. Webb, A., (1999) Statistical Pattern Recognition, Oxford University Press) involves global estimates of the hypothesis-dependent densities in a form of Gaussian distributions, for which the corresponding mean vectors and the covariance matrices are estimated. This method starts to suffer from a lack of accuracy when actual densities become more and more non-Gaussian. On the other hand, the non-parametric K-nearest neighbor method (see e.g. Webb, A., (1999) Statistical Pattern Recognition, Oxford University Press) operates locally around a new data point and assigns to this point that hypothesis (class), which corresponds to the most frequent class possessed by its K nearest neighbors. The K neighbors themselves are selected according to a Euclidean distance in the space of tests. The K-nearest neighbor method does not use any functional form for density, but has a few drawbacks such as a lack of probabilistic interpretation and the sensitivity to the choice of the K parameter (a small K may not be sufficient for making a class assignment, but a large K may involve a large local region where the density estimate will be smeared).

The diagnostics problem provides a practical application in which the global and local estimates would naturally complement to each other, and one really needs to integrate them into a unified prediction algorithm. The DBA effectively accomplishes this task.

3.1.1.1.1 Global Estimation of Density in the DBA

In the solution provided herein, the global estimate of the hypothesis-conditional density p(x/Hₖ), k=1, . . . , N is important for revealing essential statistical dependencies between tests, which is only possible when all data is used. The global estimation is helped by the fact that the realistic distributions for the gene expressions are usually single-peak distributions (“core-and-tails” PDFs). This fact was confirmed on a large number of cases since the visualization tools provided herein allow for automated visualization of various scattering plots in 2D and 3D as well as ND (via parallel coordinates)

The global estimate of hypothesis-conditional density p(x/Hₖ), k=1, . . . , N is sought in the form of a guaranteeing model of concentric ellipsoids (see FIG. 2).

The probabilistic measure of each q-th inter-ellipsoidal layer for each hypothesis Hₖ is denoted as α_{q,k}:

α_{q,k}=Pr{xεE_{q,k}∩E_{q-1,k}}, q=1, . . . , Q, E₀=E₁ (6)

and the probabilities {α_{q}} satisfy the constraint

$${\sum }_{q = 1}^{Q} {α}_{q} = \overline{α} \qquad ( 7 )$$

where \overline{α} is the guarantying probability of the entire ellipsoidal set, which is associated with removing the outliers in the hypothesis-conditional densities p(x/Hₖ), k=1, . . . , N. A practical recommendation here is to use \overline{α}→1, e.g. \overline{α}=0.95 as a standard (this number corresponds to an approximate level of the expected sensitivity/specificity of the screening test).

In Eq. (6) the m -dimensional ellipsoid E_{q,k} for each hypothesis Hₖ is defined as follows

$${E}_{q , k} = \{ x : {( x - {m}_{x , k} )}^{T} {P}_{x , k}^{- 1} ( x - {m}_{x , k} ) \leq {μ}_{q , k}^{2} \} \qquad ( 8 )$$

where the m×1 vector x is the argument in the space of tests, the m×1 vector m_{x,k} is the mean (center) of each ellipsoid, the m×m matrix P_{x,k} is the ellipsoid's covariance matrix and the scalar μ²_{q,k} defines the size of the q-th ellipsoid.

Correspondingly, the density estimate is calculated via the following formula:

\mathring{p}(x/Hₖ)=α_{q,k} if xεE_{q,k}∩E_{q-1,k}(E_{0,k}=E_{1,k}), k=1, . . . , N (9)

The guaranteeing model of the concentric ellipsoids is a generalization of the conventional Gaussian model. Indeed, in the case of Gaussian model for each hypothesis Hₖ and for each q-th layer in Eqs. (6)-(8) the parameters α_{q,k} and μ²_{q,k} would be related via the standard formulas for the n-dimensional Gaussian distribution. Unlike the conventional Gaussian model, the guaranteeing model of Eqs. (6)-(8) is adjusted (via stretching of ellipsoids) to the non-Gaussian nature of the test distributions. The guaranteeing nature of the ellipsoidal model comes from the following two facts: 1) the theorem (see Shannon, (1948) system Technical Journal, 27,:379-423 and 623-656) the entropy is a Gaussian;” and, 2) each ellipsoid associated with an instantaneous Gaussian is optimally smeared so that its entropy is increased. The latter directly leads to increasing the entropy over the hypotheses, expressed via their a posteriori probabilities {p(H₁/x), p(H₂/x), . . . , p(H_{N}/x)}:

$$H = - {\sum }_{k = 1}^{N} p ( {H}_{k} / x ) log [ p ( {H}_{k} / x ) ] \qquad ( 10 )$$

The computational convenience of the ellipsoidal model of Eqs. (6)-(8) consists in the fact that an operation with this model in Eq. (9) is not ill-conditioned, as would be an operation of computing the value of the conventional Gaussian density in a high-dimensional space with correlated features.

3.1.1.1.1.1 Evaluating the Guaranteeing Model of Concentric Ellipsoids

Here the algorithm for evaluating the guaranteeing model of concentric ellipsoids represented by Eqs. (6)-(8) is presented. This algorithm includes three major steps.

Step 1. Evaluate the robust estimate of the mean vector and covariance matrix associated with the guaranteeing probability \overline{α}.

This robust procedure seeks to reduce the effect of outliers on the density estimation for each hypothesis Hₖ via the following conventional estimation operations with the specially designed weights wi (see e.g. Webb, A., (1999) Statistical Pattern Recognition, Oxford University Press):

$$\begin{matrix}{m}_{x , k} = \frac{{\sum }_{i ∈ {I}_{k}} {w}_{i , k} {x}_{i , k}}{{\sum }_{i ∈ {I}_{k}} {w}_{i , k}} , k = 1 , … , N & ( 11 ) \\ {P}_{x , k} = \frac{{\sum }_{i ∈ {I}_{k}} {w}_{i , k}^{2} ( {x}_{i , k} - {m}_{x , k} ) {( {x}_{i , k} - {m}_{x , k} )}^{T}}{{\sum }_{i ∈ {I}_{k}} {w}_{i , k}^{2} - 1} , k = 1 , … , N & ( 12 )\end{matrix}$$

In Eqs. (11) and (12) the m×1 vector x_{i,k} (a transposed row of the test matrix X) corresponds to the i -th patient in the k -th class (hypothesis). Also, in Eqs. (11) and (12), a set of indices Iₖ, k=1, . . . , N is selected from a set all patients who are included in the training set and who are assigned a hypothesis Hₖ as a diagnosis Dᵢ:

Iₖ={iL Dᵢ=Hₖ, i=1, . . . , n}, k=1, . . . , N (13)

The weights w_{i,k} in Eqs. (11) and (12) are defined as

$${w}_{i , k} = \frac{{w}_{i , k ( {μ}_{i , k} )}}{{μ}_{i , k}} , k = 1 , … , N \qquad ( 14 )$$

where μ_{i,k} is the ellipsoid-dependent distance

$${μ}_{i , k} = {[ {( {x}_{i} - {m}_{x , k} )}^{T} {P}_{x , k}^{- 1} ( {x}_{i} - {m}_{x , k} ) ]}^{\frac{1}{2}} \qquad ( 15 )$$

The scalar Gaussian decay function w_{i,k} (μ_{i,k}), which reduces the contributions of the outliers, is defined as follows

$${w}_{i , k} ( μ ) = \{ \begin{matrix}μ & if & μ \leq {μ}_{0} \\ {μ}_{0} exp \{ - \frac{1}{2} {( μ - {μ}_{0} )}^{2} / {σ}^{2} \} & if & μ > {μ}_{0}\end{matrix} \qquad ( 16 )$$

The parameters μ₀ and σ are adjusted to ensure that a reduction rate of outliers corresponds the guaranteeing probability \overline{α}:

$$\frac{{\sum }_{i ∈ {I}_{k}}^{} {w}_{i , k} ( {μ}_{0} , σ )}{{n}_{k}} = \overline{α} \qquad ( 17 )$$

where nₖ is the number of records associated with the hypothesis Hₖ The evaluation of the mean vector m_{x,k} and the covariance matrix P_{x,k} via Eqs. (11) and (12) is an iterative process in which the weights w_{i,k} are updated via Eqs. (14)-(17). This process is repeated until convergence.

Step 2. Build a guaranteeing model of concentric ellipsoids.

The guaranteeing nature of the ellipsoidal model of Eqs. (6)-(8) follows from the fact that the confidential intervals (CI) are used for all statistical characteristics involved and a minimax algorithm for calculating the “worst” combinations of those characteristics in terms of smearing the density estimates is employed . Given the fact that the minimax algorithm is used, which “over-guarantees” the solution, Cis can be computed via the approximate formulas, which are well verified in practice (see, e.g. Motulsky, H., (1995) Intuitive Biostatistics, Oxford University Press).

For reference, the Cl-bounded estimates of the elements of the mean vector, the covariance matrix and the probability for the ellipsoidal sets are provided. For simplicity, the indices associated with the vector or matrix and the hypotheses are omitted.

The actual mean m for each element of the mean vector m_{x,k} can be bounded by the following CI (see, e.g. Motuisky, H., (1995) Inituitive Biostatistics, Oxford University Press)

CI{\hat{m}−z*\hat{σ}≦m≦\hat{m}+z*\hat{σ}} (18)

In Eq. (18) three values are used to construct a confidence interval for m: the sample mean \hat{m} defined by Eq. (11) (\hat{m} is a corresponding element of the mean vector m_{x,k}), the sample value of the standard deviation \hat{σ} defined by Eq. (12) (\hat{σ} is a root-squared element of the covariance matrix P_{x,k}) and the value of z* (which depends on the level of confidence and is the same as in Eq. (21)).

The Monte-Carlo approach is used to account for variability of the actual covariance matrix due to finite sample. This approach is based on the use of the classical Wishart distribution as a generalization of the χ²-square distribution (see, e.g.Motulsky, H., (1995) Intuitive Biostatistics, Oxford University Press):

$$p ( S ) = \frac{1}{{Γ}_{m} ( n / 2 ) {\vert \hat{P} \vert}^{\frac{n}{2}}} {( \frac{n}{2} )}^{\frac{mm}{2}} etr ( - \frac{n}{2} {\hat{P}}^{- 1} S ) {\vert S \vert}^{( n - m - 1 ) / 2} \qquad ( 19 )$$

In Eq. (19), S is the m×m matrix argument of the distribution function, \hat{P} is the estimate of the covariance matrix P_{x,k} defined by Eq. (12), n is the length of the sample. Also, etr denotes the exponential of the trace and Γₘ (γ) is the multivariate gamma function

$${Γ}_{m} ( y ) = {π}^{m ( m - 1 ) / 4} {\prod }_{j = 1}^{m} Γ ( y - \frac{1}{2} ( j - 1 ) ) \qquad ( 20 )$$

The CIs of the elements of the covariance matrix P_{x,k} are computed by Monte-Carlo simulating K values of S according to the Wishart's statistics of Eq. (20) and then selecting the lower and upper bounds for all elements so that they include a certain confidential percent of (e.g. 95%) of all simulated S.

The actual probability p for each ellipsoid in Eqs. (6)-(8) can be bounded by the following CI (see, e.g. Motulsky, H., (1995) Intuitive Biostatistics, Oxford University Press)

$$CI \{ \hat{p} - {z}^{*} \sqrt{\frac{\hat{p} ( 1 - \hat{p} )}{n}} < p < \hat{p} + {z}^{*} \sqrt{\frac{\hat{p} ( 1 - \hat{p} )}{n}} \} \qquad ( 21 )$$

where \hat{p} is the estimate of the probability, n is the length of the sampling set and z* is the quantile of the Gaussian distribution (e.g. z*=1.96 for 95% CI). The probability estimate is computed as

$$\hat{p} = \frac{q}{n} \qquad ( 22 )$$

where n is the length of the sample and q is the number of realizations within the ellipsoid.

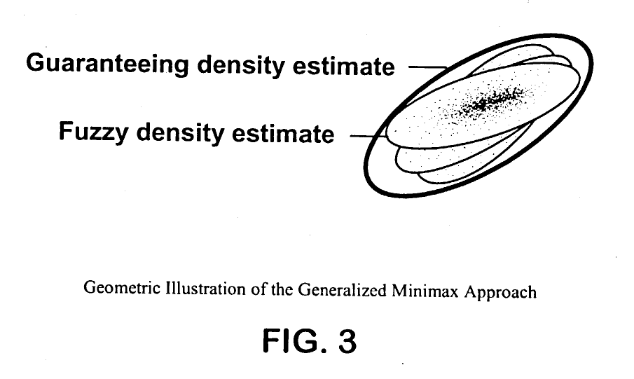

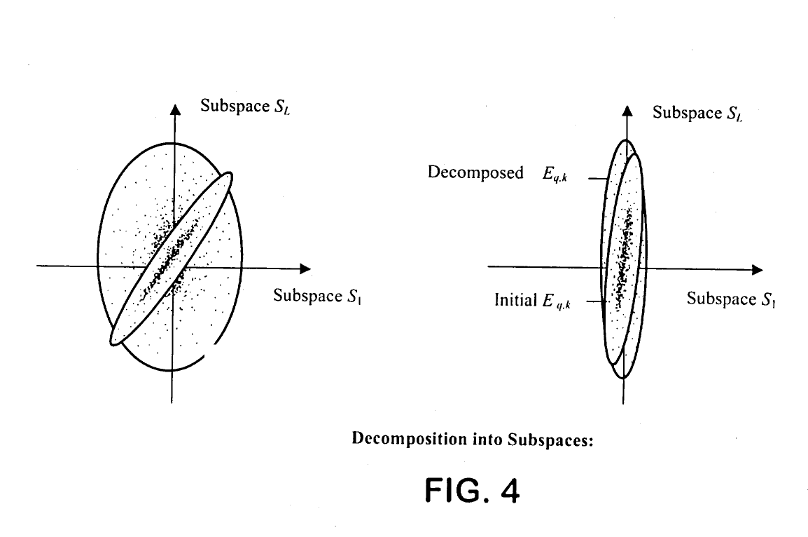

The evaluation of the guaranteeing model of concentric ellipsoids of Eqs. (6)-(8) is based on the generalized minimax algorithm (see Motulsky, (1995) Intuitive Biostastistics, Oxford University Press). First, this algorithm builds an equivalent uncertain-random model (a combination of random and bounded values) from the statistics of Eqs. (11) and (12) given the confidential intervals for their parameters as described above (see Eqs. (18)-(20)). Second, this algorithm expands each of the Q concentric m-dimensional ellipsoids E_{q,k} of Eq. (8) retaining the ellipsoid's shape and the center as defined by Eqs. (11) and (12). Thereby, the ellipsoid's sizes (parameter p in Eq. (8)) are miininally expended just to accommodate for the worst low boundary of the confidential interval, of Eq. (21) for the estimated probability \hat{p} of Eq. (22). The geometrical illustration of this algorithm is presented in FIG. 3, which shows how the fuzzy density estimate (fuzzy due to the non-zero confidential intervals for the covariance matrix) is approximated by a guaranteeing density estimate. It is important to note that this algorithm implicitly, via the probability estimate \hat{p}, accounts for the non-Gaussian nature of the densities p(x/Hₖ ), k=1, . . . , N. This is done in a guaranteeing manner, i.e. via an over-sized ellipsoid. The guaranteeing probability of each q-th ellipsoidal layer is defined by Eq. (6) as a difference of the guaranteeing probabilities of the associated larger and smaller ellipsoids, respectively.

Step 3. Identify subspaces of strongly correlated tests.

This step is especially crucial while dealing with large dimensional tests, e.g. associated with gene expression data. The guaranteeing model of the concentric ellipsoids (Eqs. (6)-(8)) is defined in the full m -dimensional space of tests. However, in the real data different tests have different levels of mutual correlations. This fact is confinned via the 2D and 3D scattering plots of gene expression data. For efficiency of dealing with the ellipsoidal model it is beneficial to decompose the full space S of tests into a few smaller subspaces S₁, . . . , S_{L}, maintaining only essential statistical dependencies. Algorithmically, the ellipsoid E_{q,k} of Eq. (8) is decomposed into sub-ellipsoids [E_{q,k}]_{S}_{ᵢ} associated with a subspace Sᵢ and corresponding to the q-th layer and k-th class (hypothesis). Algorithmically, this entails identifying those combinations of tests for which it is possible to re-orient and expand the associated sub-ellipsoid [E_{q,k}]_{S}_{ᵢ} in such a way that the following three conditions are met. First, this expanded ellipsoid includes the original ellipsoid. Second, its axes become perpendicular to the feature axes not included in the subspace Sᵢ. Third, the increase in the ellipsoids volume V is within the specified threshold \overline{ν} (e.g. 0.05-0.1):

$$\frac{V ( {\tilde{E}}_{q , k} ) - V ( {E}_{q , k} )}{V ( {E}_{q , k} )} < \overline{v} \qquad ( 23 )$$

The volume of each ellipsoid in Eq. (23) is calculated as follows

V(E)=det{P(\overline{μ}²)} (24)

where P is the ellipsoid's matrix (a scaled covariance matrix) and \overline{μ}² is a common parameter for both ellipsoids (initial and decomposed). The commonality of this parameter for both ellipsoids is needed in order to make the right-hand parts of Eq. (8) equal while attributing the differences in μ² to the ellipsoid's matrices.

FIG. 4 shows two different examples of decomposing the space of features S into two subspaces S₁and S_{L}. In the first example (left), decomposition is excessive since it is done between highly correlated subspaces. This significantly expands the final decomposed ellipsoid, i.e. increases its entropy. In the second example (right), decomposition is acceptable since the two subspaces have a low inter-correlation.

It should be emphasized that the robust estimate of the hypothesis-conditional density p(x/Hₖ ),k=1, . . . , N presented above in Steps 1-3, can be used by itself in the DBA (see Eq. (5)). This robust approximation of the density usually suffices for those patients whose test values are on the tails of distributions where diagnostics are more obvious. For those patients whose test values are in the regions closer to critical thresholds, a more accurate estimation is needed. The local estimation described in Sections 3.1.1.1.2 provides this accuracy, thus, complementing the global estimation.

3.1.1.1.1.2 Generalization for Sparse (Missing) Data

The algorithm for evaluating the guaranteeing model of concentric ellipsoids (see Section 3.1.1.1.1.1) is generalized to the case when there are missing data points in the test matrix X (sparse matrix X). This is an important generalization aimed at increasing the overall DBA's robustness while dealing with real-world data. Indeed, in the DNA microarrays data typically there is a relatively high percentage of the missing gene expressions. Also, in the diagnostics problems from gene expression data one needs to deal with the fact that not each patient has a complete set of data.

The corresponding robust algorithm to handle the missing data is a part of the iterative robust procedure of Eqs. (11)-(17). At the first iteration, in Eq. (11) for each element of the m×1 mean vector m_{x,k} the sum is taken only over those tests, which are available in the data. Similarly, in Eq. (12) for each element of the m×m covariance matrix \hat{P}_{x,k} the sum is taken only over those pairs of the tests that are both available in the data for a particular patient. In the case when each patient does not have a particular pair of tests, the covariance element corresponding those two sets is set to 0.

This approximate Gaussian distribution N {m_{x,k}, P_{x,k}} obtained from Eqs. (11) and (12) for the entire hypothesis-conditional population (k-th class) is used for generating missing data points for each i -th patient.

The m×1 vector x_{i,k} of all potential tests for the i-th patient in the k-th class is regrouped into two consecutive blocks:

$${x}_{i , k} = ( \frac{{x}_{i , k}^{A}}{{x}_{i , k}^{M}} ) \qquad ( 25 )$$

where

$${x}_{i , k}^{A}$$

is the m^{A}×1 vector of available tests for the i-th patient in the k-th class (“A” stands for the available data) and

$${x}_{i , k}^{M}$$

is the m^{M}×1 vector of missing tests for the i-th patient in the k-th class (“M” stands for the missing data). Correspondingly, the first two momenta of the approximate Gaussian distribution N {m_{x,k}, P_{x,k}} are regrouped as follows

$${m}_{x , k} = ( \frac{{m}_{x , k}^{A}}{{m}_{x , k}^{M}} ) , {P}_{si , k} = ( \frac{{P}_{x , k}^{A}}{{P}_{x , k}^{M , A}} \frac{{P}_{x , k}^{A}}{{P}_{x , k}^{M}} ) \qquad ( 26 )$$

One can construct the following observation model for the incomplete data, which shows how the available data depend on the entire set of potential tests (available and missing) for each i-th patient:

$${x}_{i , k}^{A} = ( {I}_{{m}^{A} \times {m}^{A}} \vert {0}_{{m}^{A} \times {m}^{M}} ) ( \frac{{x}_{i , k}^{A}}{{x}_{i , k}^{M}} ) \qquad ( 27 )$$

Having the statistical observation model of Eqs. (26) and (27) it is possible to employ the conventional Bayesian approach (see e.g. Gelb, A., ed., (1989) Applied Optimal Estimation, The MIT Press, Cambridge) and calculate the a posteriori distribution

$$p ( {x}_{i , k}^{M} / {x}_{i , k}^{A} ) ,$$

i.e. the distribution of

$${x}_{i , k}^{M}$$

(missing data) given the observations of x^{A}_{i,k} (available data). Under assumption that the a priori distribution

$$p ( {x}_{i , k}^{M} ) = N \{ {m}_{x , k} , {P}_{x , k} \}$$

is Gaussian and due to the fact that the observation model of Eq. (27) is linear, the a posteriori distribution

$$p ( {x}_{i , k}^{M} / {x}_{i , k}^{A} )$$

will be Gaussian too. The algorithmic form of the Bayesian approach in this particular case can be expressed via the well-known Kalman Filter (see e.g. Gelb, A., ed., (1989) Applied Optimal Estimation, The MIT Press, Cambridge and Malyshev, V. V. et al. (1992) Optimization of Observation and Control Processes, AIAA Education Series, 349 p.):

$$\begin{matrix}{m}_{xi , k}^{M / A} = {m}_{x , k}^{A} + {P}_{xi , k}^{M / A} {Ξ}_{{η}_{i}}^{- 1} ( {x}_{i , k}^{A} - {m}_{x , k}^{A} ) \\ {P}_{xi , k}^{M / A} = {P}_{x , k}^{M} - {{P}_{x , k}^{M , A} ( {Ξ}_{{η}_{0}} + {P}_{x , k}^{A} )}^{- 1} {P}_{x , k}^{A , M}\end{matrix} \qquad ( 28 )$$

where

$${m}_{xi , k}^{M / A}$$

is the a posteriori m^{M}×1 vector of mathematical expectation for each i-th patient and

$${P}_{xi , k}^{M / A}$$

is the a posteriori m^{M}×m^{M} covariance matrix for the m^{M}×1 vector

$${x}_{i , k}^{M}$$

of missing tests for each i-th patient. Also, the m^{M}×m^{M} matrix Ξ_{ηi} is the regularization matrix. In practical problems, the matrix Ξ_{ηi} is a covariance matrix of the additive measurement noise, associated with errors in measuring the test values in medical laboratories. When the observations are ideally precise then the elements of the matrix Ξ_{ηi} can be set to small numbers

$$( {Ξ}_{{η}_{i}} • {P}_{x , k}^{A , M} )$$

for the regularization purpose in order to use the Kalman Filter of Eqs. (28).

The a posteriori distribution

$$p ( {x}_{i , k}^{M} / {x}_{i , k}^{A} ) = N \{ {m}_{x , k}^{M / A} , {P}_{x , k}^{M / A} \}$$

serves as a fuzzy substitute for missing data points

$${x}_{i , k}^{M} .$$

Correspondingly, the missing value is substituted by a random generalization from the above distribution:

$${x}_{i , k}^{M} = A ξ + {m}_{x , k}^{M / A} \qquad ( 29 )$$

Here, ξ is a random realization of the m^{M}×1 standard Gaussian vector with the zero mathematical expectation and the unity covariance matrix (all diagonal elements are equal to 1 and the off-diagonal elements are equal to 0), A is the Choleski decomposition of the a posteriori covariance matrix

$${P}_{x , k}^{M / A}$$

so that

$$AA = {P}_{x , k}^{M / A} .$$

It should be noted that establishing the correlations between all pairs of tests facilitates a narrower spread of the values

$${x}_{i , k}^{M} .$$

After a new test matrix X is formed, it is used on the next iteration in the extended iterative procedure of Eqs. (14)-(17), Eq. (28). This involves the update of the statistics N {m_{x,k}, P_{x,k}} and, correspondingly, the a posteriori statistics

$$N \{ {m}_{x , k}^{M / A} , {P}_{x , k}^{M / A} \} .$$

The updated a posteriori statistics are used via Eq. (29) for generating new realizations of the missing data points in the test matrix X for Eqs. (11) and (12). It is important to note that the fuzzy nature of the generated missing data points is further accounted for in the diagnostics (classification) process.

3.1.1.1.1.3 Generalization for Multiple-Set Densities

The Gaussian-like approximation of the global density in a form of guaranteeing concentric ellipsoids (see Section 3.1.1.1.1). can be generalized to its multiple-set version. This generalization is useful in the cases when the hypothesis-conditional density p(x/Hₖ ), k=1, . . . , N can be thought as a set of different Gaussian-like densities. The multiple-set density can be formalized via a convolution

$$p ( x / {H}_{k} ) = {\sum }_{j = 1}^{J} {ρ}_{j} ( x / {H}_{k} ) {p}_{j} ( x / {H}_{k} ) \qquad ( 30 )$$

where the hypothesis-conditional density pⱼ (x/Hₖ) is associated with the j-th set and ρⱼ (x/Hₖ) is a probabilistic measure governing (stochastically) a choice of the j-th set.

A practical approach to constructing the multiple-set model of Eq. (30) is based on cluster analysis. The clustering techniques are described in Section 3.1.1.2. In this particular case samples (patients) in each k-th class (diagnosis) are clustered in an attempt to identify L most separated clusters in the space of features (tests). When these clusters exist one can split a space of features x in J regions Ω_{j,k}, j=1, . . . , J associated with each cluster. The boundaries of the regions Ω_{j,k}, j=1, . . . , J can be chosen in an ellipsoidal form similar to Eq. (8) given the mean vector and the covariance matrix for x in each j-th set.

The choice of the probabilistic measure ρⱼ (x/Hₖ) can be the following:

$${ρ}_{j} ( x / {H}_{k} ) = α \frac{{ⅆ}_{j}}{{\sum }_{j = 1}^{j} {ⅆ}_{j}} \qquad ( 31 )$$

where

$${d}_{j} = {[ {( x - {m}_{xj} )}^{T} ( x - {m}_{xj} ) ]}^{\frac{1}{2}}$$

is the distance from a particular x point the region Ω_{j,k}, j=1, . . . J. Also, in Eq. (31), α is a scalar which normalizes the density p(x/Hₖ):

$$p ( x / {H}_{k} ) : {\int }_{x ∈ {R}^{m}} p ( x / {H}_{k} ) ⅆ x = 1.$$

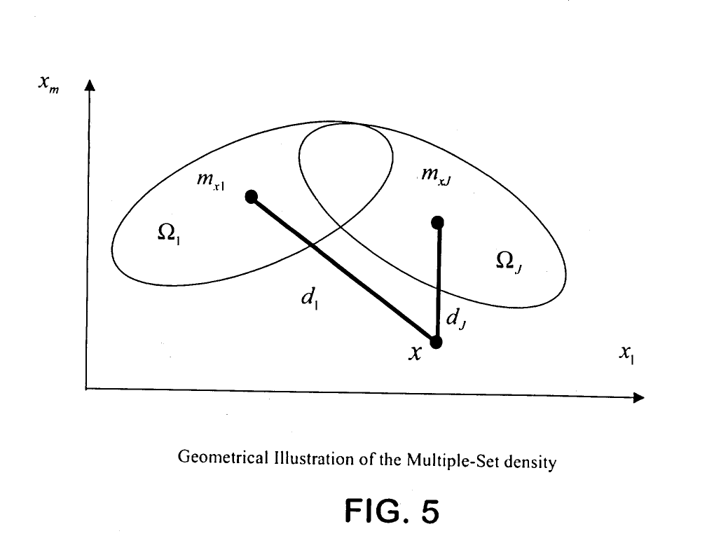

Geometrical illustration of the multiple-set density is provided in FIG. 5.

3.1.1.1.2 Local Estimation of Density in the DBA

From a practical point of view (medical diagnostics), the important element of the DBA for interpreting the “local” aspect of the density estimation involves a statistical generalization of the threshold principle currently used in medical practice for diagnostics. According to this principle the “hard” test values are established (e.g. by the World Health Organization or other medical associations) for the use as thresholds in detecting a certain disease. The key advantage of the statistical generalization consists in the fact that the DBA uses a system of “soft thresholds” and, thus, detects a more complex hidden pattern of a disease in the space of multiple tests. The search for these patterns is localized around the “hard thresholds”, i.e. in the regions where the accurate diagnostics are critical.

From a mathematical point of view, the DBA for local density estimation presents a principally different method compared with the state-of-the-art methods, e.g. K-nearest neighbor method or kernel methods (see e.g. Webb, A., (1999) Statistical Pattern Recognition, Oxford University Press). Three two major innovations of the DBA for estimating density locally are the following:

1) Soft thresholds for diagnostics

2) Definition of neighborhood in the space of critical distances to thresholds

3) Statistical discrete patterns of neighbor counting

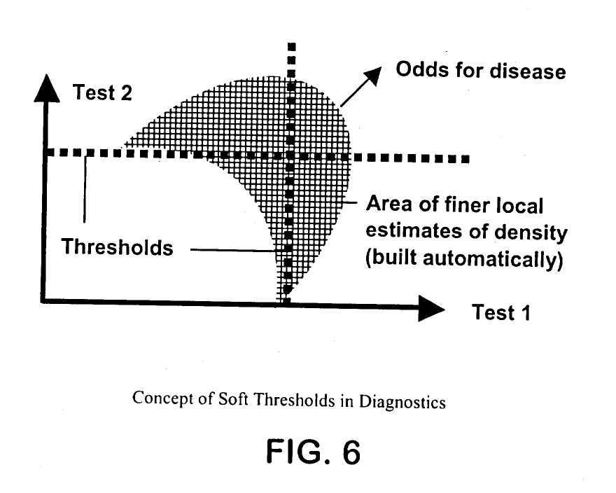

FIG. 6 presents a general idea of the concept of soft thresholds, which is formalized via a novel way of estimating density locally. In other words, a probabilistic measure around the hard thresholds is defined in order to better formalize the statistical nature of the odds for a particular disease.

The local estimation of density entails computing a distance from the dataset of tests for a new patient x_{new} to the dataset of neighbors x_{i,k} where i counts diagnosed patients and k identifies a diagnosis (class). The global density estimation (see Section 3.1.1.1.1) provides important reference information for the local density estimation. This is due to the knowledge of statistical dependencies between the tests, which are estimated globally and are formalized in the form of a guaranteeing model of concentric ellipsoids represented by Eqs. (6)-(8). This knowledge contributes to a better definition of distance between the data points in the local area.

This distance between x_{new} and x_{i,k} is defined as follows

$${d}_{i , k} = {( {x}_{new} - {x}_{i , k} )}^{T} {P}_{x , k}^{- 1} ( {x}_{new} - {x}_{i , k} ) \qquad ( 32 )$$

where P_{x,k} is the m×m covariance matrix for the k-th class. This matrix globally (i.e. using the global estimate of density on the entire data in the class) transforms the distance space in such a way that the distance between neighbors accounts for the observed correlations in the tests values (for the given class).

The latter fact is not difficult to prove. First, the space of features can be transformed into an uncorrelated set of features z:

z_{i,k}=A⁻¹(x−m_{x,k}) (33)

where m_{x,k} is the m×1 mean vector for the k-th class and A is the Choleski decomposition of the covariance matrixp, P_{x,k} so that AA=P_{x,k}. Second, in the transformed space of the uncorrelated features z, the distance d_{i,k} can be expressed in a form, invariant to the mean vector mX,k, and this directly leads to Eq. (32):

i d_{i,k}=∥z_{new}−z_{i,k}∥²=(z_{new}−z_{i,k})^{T}(z_{new}−z_{i,k})=∥A⁻¹(x_{new}−m_{x,k})−A⁻¹(x_{i,k}−m_{x,k})∥²=∥A⁻¹(x_{new}−x_{i,k})∥²=(x_{new}−x_{i,k})^{T}[AA]⁻¹(x_{new}−x_{i,k})=(x_{new}−x_{i,k})^{T}P_{x,k}⁻¹(x_{new}−x_{i,k}) (34)

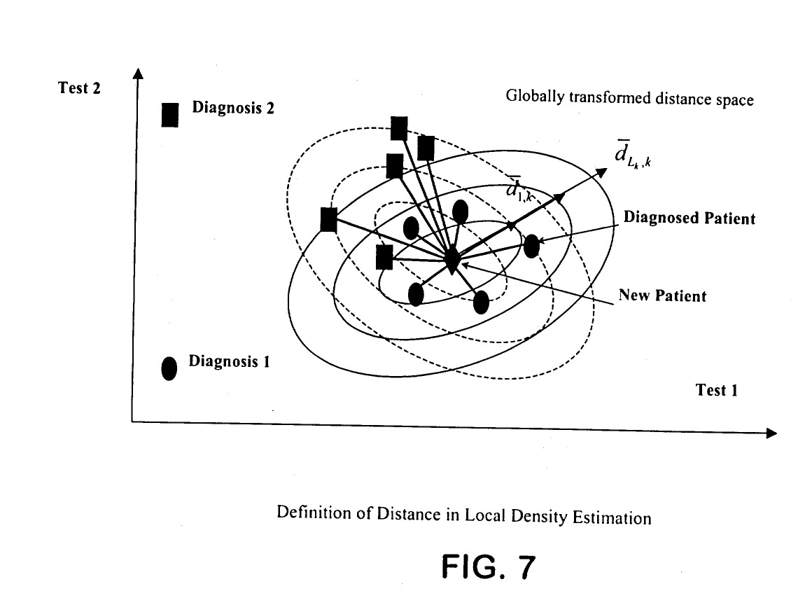

FIG. 7 illustrates the transformation of a local distance space around a new patient, given the global estimates of density. Two diagnoses (classes) and two tests are shown. The ellipsoidal contour lines indicate how the tests are inter-dependent in each class. A sequence {d_{1,k}}(l=1, . . . L) for each k-th class discretizes the transformed distance space in layers.

An important element of the local density estimation is a statistical discrete neighbor counting pattern. This pattern is defined in a form of counting neighbors in the distance layers for each class: {C_{1,k}}, l=1, . . . , Lₖ. The integer C_{1,k} is the number of patients diagnosed with the k-th diagnosis whose tests are distanced from the new patient's tests within the l-th distance layer for the k-th class:

$${C}_{l , k} = {\sum }_{i}^{{n}_{k}} {ϑ}_{l , i , k} , {ϑ}_{l , i , k} = \{ \begin{matrix}1 & if {\overline{d}}_{l - 1 , k} < {d}_{i , k} \leq {\overline{d}}_{l , k} , {\overline{d}}_{0 , k} = 0 \\ 0 & otherwise \end{matrix} \qquad ( 35 )$$

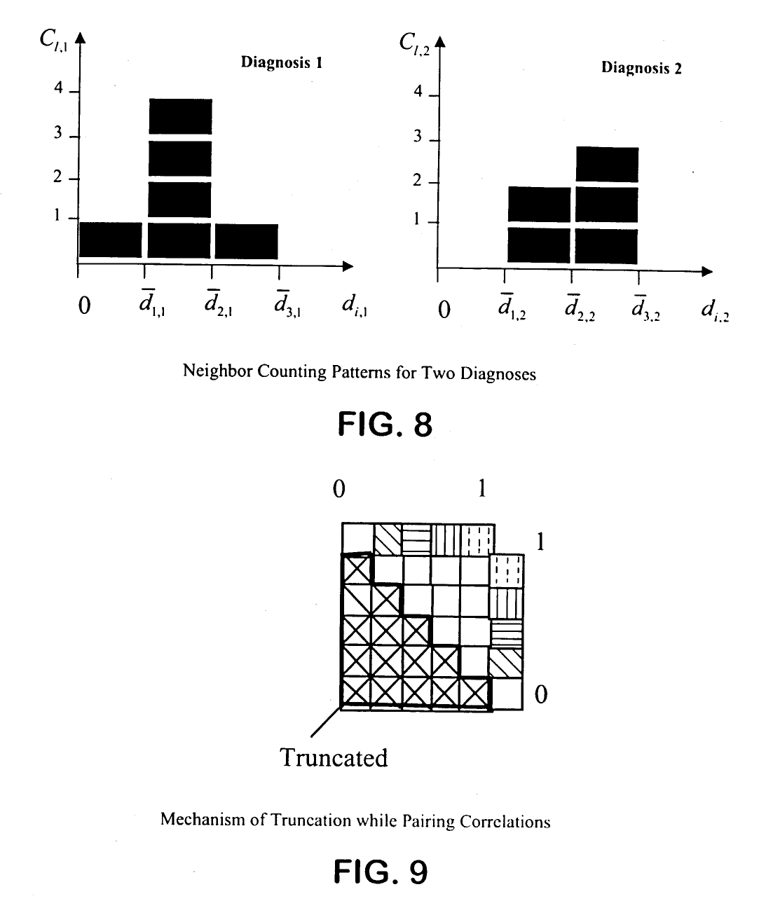

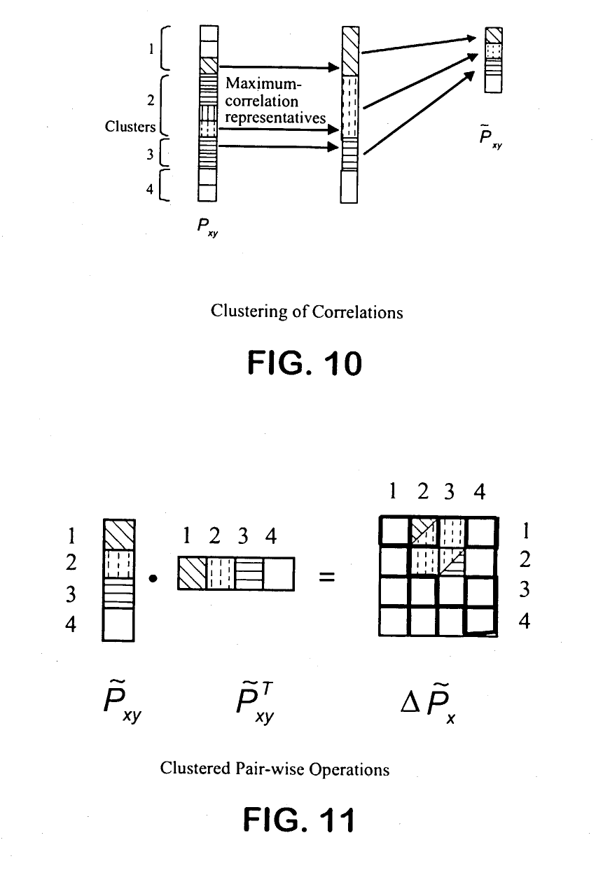

FIG. 8 shows a geometrical illustration of the neighbor counting patterns for two diagnoses (diagnoses 1 and 2). Note that these patterns correspond to FIG. 7.

Using Eq. (35) with Eq. (32), one can generate the observed discrete neighbor counting patterns for any new patient whose tests values are x_{new}. They are similar to those shown in FIG. 8, i.e. they are generated for each k-th class (diagnosis) and each l-th layer of the class-dependent distance of Eq. (32). The discrete neighbor counting patterns can be considered as a transformed set of features, introduced to handle local aspects of the classification problem.

Correspondingly, the problem of estimating (locally) the hypothesis-conditional densities p(x/Hₖ ), k=1, . . . , N is transformed into a problem of determining probabilistic measure on the discrete neighbor counting patterns {C_{1,k}}l=1, . . . , Lₖ, k=1, . . . , N.

3.1.1.2 Clustering Techniques

Although the DBA generates the predictive models from gene expression data, in the latter case additional challenges arise. This is mainly due to the fact that the gene expression data is defined in a very high dimension of features, the number of which can reach thousands. A typical example of gene expression data for the toxicity studies involves about 9,000 genes. In this case clustering techniques are necessary in order to deal with very high dimension of gene expressions in DNA microarrays applications. Clustering entails that the genes with similar gene expression patterns can be grouped into clusters, which represents a common pattern for the genes clustered together. This reduces the space of features to tens or hundreds, opening the way for the decomposition techniques (such as global-local estimation of density) described in Section 3.1.1.1.

But, clustering itself is a challenging mathematical problem for the cases when the number of objects for clustering exceeds 2-3 thousands. The state-of-the-art methods for clustering (see e.g. Webb, A., (1999) Statistical Pattern Recognition, Oxford University Press) can be split into two basic groups: 1) hierarchical or matrix methods; and, 2) iterative methods.

The hierarchical methods (such as single-link method, complete-link method, sum-of-squares method, and general agglomerative algorithm) (see e.g. Webb, A., (1999) Statistical Pattern Recognition, Oxford University Press) are extremely expensive in memory and slow in speed since they require the calculation and O(m²) operations with the full distance matrix m×m between all m features (again, m reaches thousands). For example, among the time-consuming operations are the tree-like operations, which are needed in order to perform the hierarchical clustering and evaluate the hierarchical tree, which shows how objects are related to each other. Moreover, when clustering is a part of the predictive algorithm the multiple clustering operations are needed, which makes the matrix clustering very complex and practically infeasible in high-dimensional problems.

The iterative methods (such as various versions of the K-means method) (see e.g. Webb, A., (1999) Statistical Pattern Recognition, Oxford University Press) offer an efficient computational alternative, which does not require the use of any matrix construction, i.e. all operations are O(m) . Indeed, the method just follows the principle of assigning an object to the closest cluster and these assignments are done iteratively, before convergence (no more change in cluster assignment) is reached. However, the iterative methods have a drawback of poor convergence, i.e. the iterative procedure can be easily trapped in a local minimum. Practically, this means that even well-separated data points can be grouped into a single cluster or, vice versa, the data points making a perfect cluster can be split into two or more clusters. All methods aimed at improving the K-means iterative procedure are heuristic to a large degree and do not guarantee convergence especially in the case of high dimensionality.

Provided herein is a principally different way of clustering which utilizes the advantages of matrix methods (accuracy) and iterative methods (efficiency). This is achieved via the concept of Correlation Wave (CW). The main idea of CW consists in the decomposing of the global clustering problem (associated with the use full-matrix operations) into a sequence of local problems (associated with the of use much smaller sub-matrices). The local problems are seamlessly linked with each other so that all essential correlations are retained and no information is lost.

The CW-based clustering algorithm is developed to handle realistic situations in the gene expression analysis, which are typically characterized with a high level of variability and overlaps in the data. Correspondingly, a rigorous statistical treatment of these situations (data variability and overlaps) is offered via a robust stochastic clustering. As a result of this, the robust stochastic clustering algorithm provided herein generates a reliability measure for gene assignments to clusters. The stochastic nature of the clustering algorithm and its efficient computational engine based on the CW decomposition are highly intertwined, since a probabilistic measure is used to link local matrix-based clustering problems.

The Nonlinear Recursive Filter (see Padilla, et. Al (1995) Proceedings of the SPIE on Spaceborne Interferometry, 2477: 63-76. and Malyshev, V. V. et al. (1992) Optimization of Observation and Control Processes, AIAA Education Series, 349 p.) is used as an clustering algorithm for detecting the closest distances between objects. The CW (Correlation Wave) algorithm adds the desirable efficiency to the NRF by exploiting the sparsity of its covariance matrix. It makes it possible to operate on small fragments of the covariance matrix and seamlessly link them with each other. In other words, the CW strategy makes it possible to retain the accuracy of the full-matrix operation but eliminate the cost of dealing with a large covariance matrix.

The clustering problem can formalized by the following state-space nonlinear model

xᵢ=ƒᵢ₋₁(xᵢ₋₁)+Fᵢ₋₁(xᵢ₋₁)ξᵢ₋₁, i=1, . . . , N (36)

yᵢ=g(xᵢ)+ηᵢ (37)

Eq.(36) describes the nonlinear dynamics of building links between objects and Eq. (37) represents the nonlinear measurement model. Correspondingly, the notations are: x for n [symbol] state-vector formalizing a cluster assignment for each object (feature) as a number 1, 2, 3 etc. (treated as a continuous number), ξ for n_{ξ} [symbol] disturbance vector (modeled as a random process with zero mean and the covariance matrix [symbol] _{ξ}), y for m [symbol] measurement vector, η for m [symbol] vector of measurement noise (with zero mean and the covariance matrix [symbol] _{η}). Also, in Eq. (36) and Eq. (37), the nonlinear models for dynamics and measurements are formalized by the nonlinear functions ƒ(·) and g(·), correspondingly. Additional nonlinearity F(·)−n [symbol] n_{ξ} formalizes the projection of the additive factors ξ into the space of the state-vector x.

Note that the NRF will directly account for the nonlinearities ƒ(·) and g(·) via utilization of their higher-order derivatives. A simpler linearized model will also be used. It should be emphasized that the filtering algorithms will actually exploit linearization in the vicinity of the current estimates. The linearized form of Eq. (36) and Eq. (37) is written as

xᵢ=Aᵢ₋₁xᵢ₋₁+Bᵢ₋₁uᵢ₋₁+Fᵢ₋₁ξᵢ₋₁+Dᵢ₋₁ζᵢ₋₁, i=1, . . . , N (38)

yᵢ=Cᵢxᵢ+ηᵢ+Gᵢ [symbol] ᵢ (39)

Eq. (38) describes the linearized dynamics and Eq. (39) represents the linearized measurements. Note that for simplicity Eqs. (38) and (39) use the same (as in Eqs. (36) and (37)) notations x and y, for the state-vector and measurement vector assuming however the model errors due to neglecting higher-order nonlinear effects are included. Moreover, Eqs. (38) and (39) are associated with the perturbations with respect to the reference values. Note that the reference values of x, y, and u are added to the perturbed values of x, y, and u to make those values similar (within the error of neglecting higher-order nonlinear effects) to the values of x, y, and u in Eqs. (36) and (37). Also, in Eq. (38) and Eq. (39), the corresponding system matrices A−n [symbol] n, F−n [symbol] n_{ξ}, and C−m [symbol] n are obtained via linearization of the original nonlinear models about the reference values for dynamics and measurements (again, for simplicity we use the same notations for the matrices after linearization as in the original nonlinear models).

Using the dynamics model of Eq. (36) and the measurement model of Eq. (37), the NRF was developed for the nonlinear filtering problem (see Padilla, et. Al (1995) Proceedings ofthe SPIE on Spaceborne Interferometry, 2477: 63-76. and Malyshev, V. V. et al. (1992) Optimization of Observation and Control Processes, AIAA Education Series, 349 p.). Its main idea is based on the transformation of coordinates in the estimation problem, while processing thej-th component of the measurement vector yᵢ at the i-th step:

$${y}_{ij} = ( 1 \vert {0}_{n \times l} ) ( \frac{{g}_{ij}}{{x}_{ij}} ) + {η}_{ij} , i = 1 , … , N ; j = 1 , … , M \qquad ( 40 )$$

In Eq. (40) the new augmented state-vector

$${X}_{ij} = ( \frac{{g}_{ij}}{{x}_{ij}} ) \qquad ( 41 )$$

consists of the measurement mean value gᵢⱼ=E[yᵢⱼ/x] and of the state-vector xᵢⱼ which corresponds to the current measurement component yᵢⱼ;ηᵢⱼ is the j-th component of the measurement error vector ηᵢ. In these new coordinates Eq. (40) becomes linear and has a particular structural advantage that is exploited in the filter design. Namely, now all measurement nonlinearities become isolated in the “dynamics” of the system for the augmented state-vector Xᵢⱼ. Note that these “dynamics” are defined between processing two measurement components and actually formalize the correlation mechanism between the original state vector xᵢⱼ and the measurement nonlinearity gᵢⱼ (xᵢⱼ).

Given the transformation of Eq (40), the main NRF computations consist of two steps: 1) analysis (nonlinear); and, 2) update (linear). These steps are realized at each time-step i to process each single component of the measurement vector yᵢ. Note that between the steps i ordinary prediction equations for the system's dynamics (linear or nonlinear) take place making, thus, a third (prediction) step of the NRF.

At the j-th analysis step(corresponding to the i-th epoch), the extended state-vector is propagated from the (j−1)-th to thej-th component of the measurement vector yᵢ. This procedure is a conventional problem of nonlinear statistical analysis:

ŷᵢⱼ=E[gᵢⱼ(xᵢⱼ)/y^{i,j-1}]

\hat{x}ᵢⱼ=\hat{x}_{i,j-1}

\hat{P}_{y}_{ᵢⱼ}=Cov [gᵢⱼ(xᵢⱼ)/y^{i,j-1}] (42)

\hat{P}_{xy}_{ᵢⱼ}=Cov[xᵢⱼgᵢⱼ(xᵢⱼ)/y^{i,j-1}]

\hat{P}ₓ_{ᵢⱼ}=\hat{P}ₓ_{_{i,j-1}}

where P stands for the corresponding blocks (y, xy, x) of the a priori covariance matrix for the extended state-vector of Eq. (4 1), and E[·] and Cov[·] are the operators of the mathematical expectation and covariance matrix, respectively. It is important to note that the operators of Eq. (42) are open to the choice of the method of statistical analysis. One can make this choice depending on how much of the problem's nonlinearity needs to be retained. For example, the operators of Eq. (42) can be evaluated by expanding the nonlinear function gᵢⱼ(xᵢⱼ) in a Taylor series in the vicinity of the a priori estimate \hat{x}ᵢⱼ retaining as many terms as needed (usually, the second- or third-order polynomials are used). A more sophisticated and more accurate choice involves Monte Carlo simulations to estimate the operators E[·] and Cov[·]. The analysis step is the only nonlinear operation in the NRF as to treating measurement nonlinearities.

The principal assumption enabling a simple update step is approximation of the a priori probability density function (p.d.f.), p(Xᵢⱼ/y^{i,j-1}), by the Gaussian density with the momenta characteristics evaluated in Eq. (42):

$${\hat{X}}_{ij} = ( \frac{{\hat{y}}_{ij}}{{\hat{x}}_{ij}} ) , {\hat{P}}_{{X}_{ij}} = ( \begin{matrix}{\hat{P}}_{{y}_{ij}} & {( {\hat{P}}_{{xy}_{ij}} )}^{T} \\ {\hat{P}}_{{xy}_{ij}} & {\hat{P}}_{{x}_{ij}}\end{matrix} ) \qquad ( 43 )$$

Given the assumption about Gaussian distribution, the update step becomes nothing else than the linear Kalman projection (see Gelb (1989) Applied Optimal Estimation, The MIT Press, Cambridge):

$$\begin{matrix}{x}_{ij}^{*} = {\hat{x}}_{ij} + ( \frac{{P}_{{xy}_{ij}}^{*}}{{Ξ}_{{η}_{ij}}} ) ( {y}_{ij} - {\hat{y}}_{ij} ) \\ {P}_{{x}_{ij}}^{*} = {\hat{P}}_{{x}_{ij}} - {\hat{P}}_{{xy}_{ij}} {\hat{P}}_{{xy}_{ij}}^{T} ( \frac{1}{{Ξ}_{{η}_{ij}} + {\hat{P}}_{{y}_{ij}}} ) \\ {P}_{{xy}_{ij}}^{*} = {\hat{P}}_{{xy}_{ij}} ( \frac{{Ξ}_{{η}_{ij}}}{{Ξ}_{{η}_{ij}} + {\hat{P}}_{{y}_{ij}}} )\end{matrix} \qquad ( 44 )$$

Between the time-steps (epochs) the NRF uses a conventional statistical analysis in a dynamic system of Eq. (36) type:

\hat{x}ⱼ=E[xᵢ]

\hat{P}ₓ_{ᵢ}=Cov[xᵢ] (45)

where xᵢ=ƒᵢ₋₁(xᵢ₋₁)+Bᵢ₋₁(xᵢ₋₁)μᵢ₋₁+Fᵢ₋₁(xᵢ₋₁)ξᵢ₋₁

Note that the operators E[·] and Cov[·] use the a posteriori statistics of the state-vector xᵢ₋₁ (available after the NRF processes the last measurement component at the (i−1)-th epoch) and the a priori statistics of the disturbance ξᵢ₋₁. Similarly to the statistical analysis of Eq. (42), the problem of Eq. (45) can be solved by many well-known methods as was discussed above.

It should be noted, that for the linearized model of Eq. (38), the NRF becomes a conventional Linearized Extended Kalman Filter (see e.g. Gelb, A., ed., (1989) Applied Optimal Estimation, The MIT Press, Cambridge)

$$\begin{matrix}{x}_{i}^{*} = {\hat{x}}_{i} + {\hat{P}}_{i} {C}_{i}^{T} {Ξ}_{{η}_{i}}^{- 1} ( {y}_{i} - {C}_{i} {\hat{x}}_{i} ) \\ {\hat{P}}_{i} = {\hat{P}}_{i} - {\hat{P}}_{i} {{C}_{i}^{T} [ {Ξ}_{{η}_{i}} + {C}_{i} {\hat{P}}_{i} {C}_{i}^{T} ]}^{- 1} {C}_{i} {\hat{P}}_{i} \\ \hat{{x}_{i}} = {A}_{i - 1} {x}_{i - 1}^{*} + {B}_{i - 1} {u}_{i - 1} \\ {\hat{P}}_{i} = {A}_{i - 1} {P}_{i - 1}^{*} {A}_{i - 1}^{T} + {F}_{i - 1} {Ξ}_{{ξ}_{i - 1}} {F}_{i - 1}^{T} , i = 1 , … , N\end{matrix} \qquad ( 46 )$$

Note that in Eq. (46) yᵢ is the vector of all measurement components at the i-th measurement epoch. Note that with appropriate indexing, Eq. (46) can be used for recursion in measurements, i.e. for processing measurement components one at a time. But, unlike the NRF, where measurement recursion helps to overcome energy barriers in the nonlinear optimization problems, here it effects only computational efficiency by making the inverse operation [Ξ_{ηi}+Cᵢ\hat{P}ᵢCᵢ^{T]}⁻¹ scalar.

The CW for the NRF involves the development of a criterion that allows us to identify and select the active fragments of the covariance matrix for each measurement. The design of this criterion became possible after the NRF equations (Eq. (42) and Eq. (44)) were given a simple physical interpretation. In particular, a simple insight was found into the mechanism of building-up the covariance matrix during nonlinear filtering. This insight implies that the contribution ΔPₓ to the n×n a posteriori covariance matrix from each measurement is built-up from the n×1 covariance vector P_{xy} (see Eq. (44)):

$$Δ {P}_{x} = {P}_{xy} {P}_{xy}^{T} \qquad ( 47 )$$

One can easily (since it is only 1 D operation) compute the correlation vector with the elements

(K_{xy})_{q}=(P_{xy})_{q}/\sqrt{(Pₓ)_{qq}P_{y}}, (q=1, . . . , n) (48)

which has a clear physical sense: the element (K_{xy})_{q} shows how the scalar measurement component is correlated with the state vector element xᵢ. In other words, the n×1 correlation vector K_{xy} provides an important clue for identifying a local space (a subset of the state vector) to which a scalar measurement contributes. These contributions can be clustered in terms of the absolute values of correlations. [0-0.1), [0.1-0.2), [0.2-0.3), [0.3-0.5), [0.5-0.1] clusters were used as a trade-off between the resolution in correlations and the number of index operations. As will be shown below, clustering will greatly reduce the computational cost of the 2D (pair-wise) operation of Eq. (47) due to the work with essential correlations only. To identify the essential-correlations the truncation mechanism is used as shown in FIG. 9. This mechanism works with the absolute values of correlations and makes a decision whether two correlation clusters interact with each other, or not. Namely, it decides whether the multiplication of two correlations (belonging to the two correlation clusters) should be an essential value or should be truncated to zero.

To effectively exploit the truncation mechanism of FIG. 9 during the pair-wise operation of Eq. (47), a technique of the embedded clustering has been developed. This technique is based on grouping of the state vector into common clusters. In this case the vector of absolute correlation values |K_{xy}| (and, thus, the covariance vector P_{xy}) becomes grouped in the same way as the state vector. FIG. illustrates this clustering operation. After being clustered, the covariance vector P_{xy} can be collapsed into a much smaller covariance vector \hat{P}_{xy} (e.g. 10-20 times smaller if clustering is performed for each single spacecraft). In this case all elements from a cluster become represented by a single element which correlation has the maximum absolute value.

Having a collapsed covariance vector \tilde{P}_{xy} (and its correlation version \tilde{K}_{xy}) one can evaluate which pairs of clusters will yield a non-zero block in the covariance matrix according to the NRF's operation (a “coarse” version of Eq. (47))

$$Δ {\tilde{P}}_{x} = {\tilde{P}}_{xy} {\tilde{P}}_{xy}^{T} \qquad ( 49 )$$

In this way one can effectively exclude large blocks of non-correlated (or, to be more exact, non-essentially correlated) parameters. FIG. 11 illustrates this selection of the essential (colored) and non-essential (blank) blocks in the clustered covariance matrix. Note that only an upper triangular of the covariance matrix is used in this illustration (and in actual calculations). As one can see, only the blocks 1-2, 2-2, 2-3 are identified as essential.

It is important to stress that all truncations in Eq. (49) (depicted in FIG. 11) are performed in a form of logical operations involving only lists of indexes, rather than actual multiplications with real numbers.

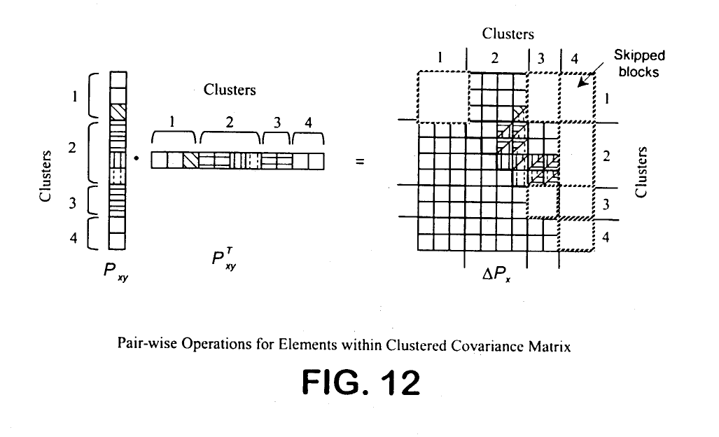

FIG. 12 illustrates the “fine” (i.e. dealing with all elements of the state vector) operation of Eq. (47). In this operation the non-essential covariance matrix blocks (identified in the “coarse” procedure) are skipped. Correspondingly, the actual pair-wise multiplications in Eq. (47) are performed only for interacting clusters, which make the blocks 1-2, 2-2, and 2-3 in the covariance matrix. Moreover, the element-by-element operations between two interacting clusters are also economized via doing pair-wise multiplications only for those pairs of indexes, which are selected as essential. As can be seen from FIG. 12, the number of these pairs is relatively small. Note that in FIG. 12, the essential covariance elements are shown as a combination of two colors corresponding to the clusters in the correlation vector K_{xy}, or similarly in the covariance vector P_{xy}. These two colors are “compatible” in the sense that they produce essential covariance element (according to the truncation mechanism depicted in FIG. 9).

All these highly economized operations dramatically reduce the computational cost of the NRF. In fact, the entire procedure of evaluating the covariance matrix tends to scale as O(m) instead of O(m²) (where m is the size of the state vector, i.e. the number of features to be clustered). The strength of the O(m) tendency depends, of course, on a particular physical system, but in high-dimensional gene expression data this tendency is strongly pronounced due to the fact that the genes appear to “work” cooperatively in large groups (clusters) associated with particular biological pathways.

4. Applications of the DBA

Stochastic Clustering for Gene Expressions

State-of-the-art clustering algorithms incorporated in current commercial software packages can be characterized by the following three points: 1) they assign a gene to one cluster; 2) they use a single deterministic distance (from a set of possible distance metrics) between genes as a measure of similarity/dissimilarity; and 3) they face tough cutoff decisions when gene expressions vary over different samples and/or are overlapped.

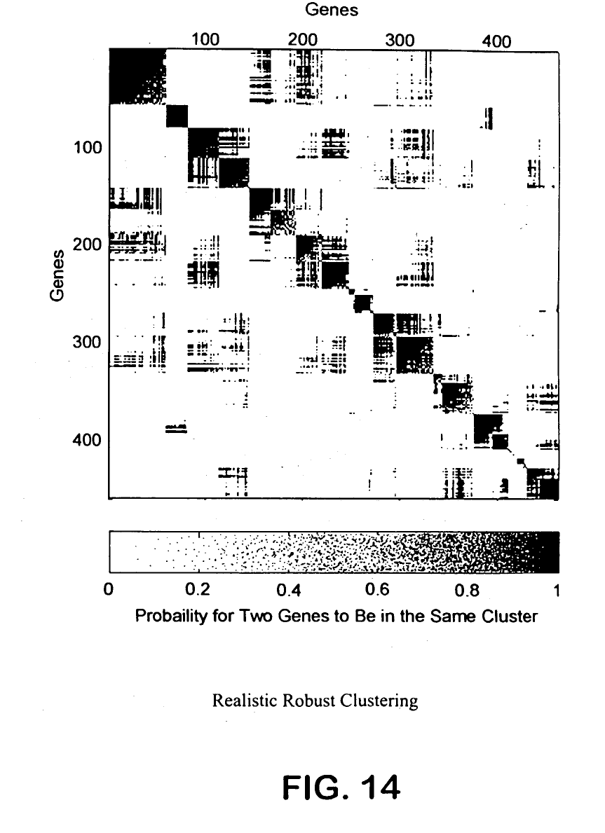

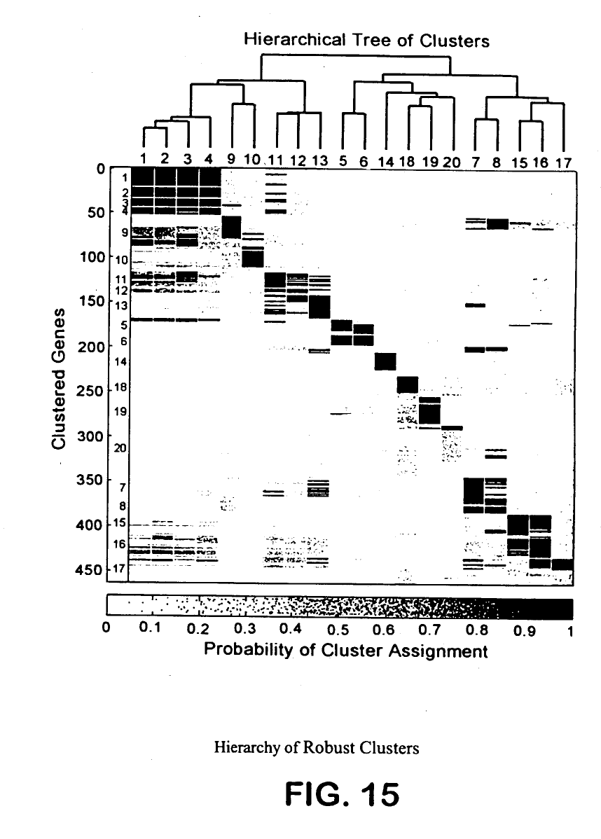

Robust clustering algorithm provided herein, alleviates these difficulties via a rigorous statistical treatment of variability and overlaps in the data. As a result of this, the robust clustering algorithm provided herein generates a reliability measure for gene assignments to clusters. FIG. 14 shows an example of realistic robust clustering. Here, the fuzziness of the clusters is due to the variability of the gene expressions over samples and overlaps in the gene expression data. Note that genes show different clustering characteristics for the given samples and conditions. Some genes cluster stably and some genes migrate between clusters. There are particular patterns of “cluster interactions.” As shown in FIG. 15, these patterns are highly correlated with the hierarchical tree of clusters (genes tend to “migrate” between similar clusters). By exposing and probabilistically handling this information (instead of hiding it through arbitrary threshold decisions), the user is provided with additional flexibility in the analysis. For example, he or she may want to investigate the “most” stable genes first.

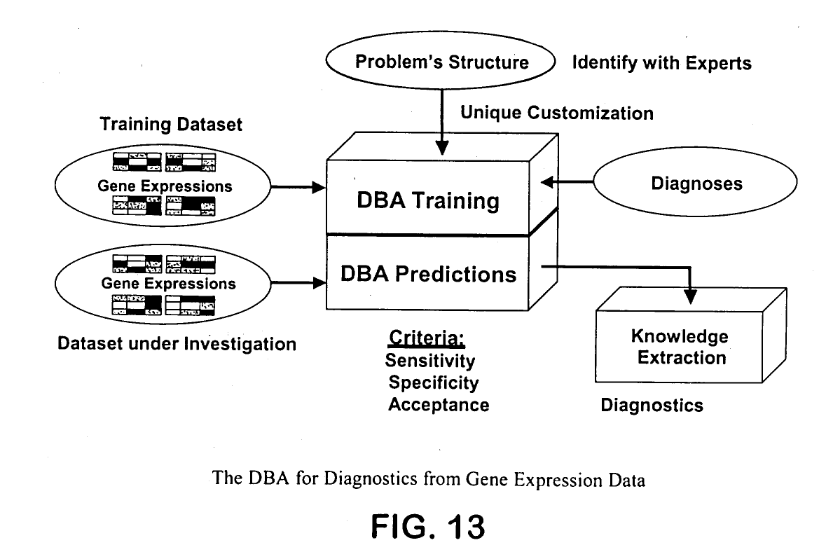

Thus, the DBA can be used for clustering (non-supervised learning) as well as for predictions (supervised learning) when the data records are labeled given additional knowledge. For example, the labels can be a disease, or a stage of disease, or any other clinical or biological information. FIG. 13 shows a schematic of how the DBA can be organized for diagnostics prediction from gene expression array data.

The following example is included for illustrative purposes only and is not intended to limit the scope of the invention.

### EXAMPLE 1

Application of the DBA for Predicting Breast Cancer Patient Outcomes From Gene Expression Data

In this example the Discrete Bayesian Algorithm (DBA) was used to predict 5-year reoccurence breast cancer outcome from gene expression data and a study was undertaken to compare the performance of the DBA technology to that of the correlation-based classification algorithm by Veer et. al. (see Laura J. van't Veer et al., January 2002, Nature, p. 530-536). For this study, the breast cancer gene expression data set used by Veer et. al was used and the predictive results obtained by the two algorithms were compared.

Gene expression signatures allowing for discrimination of breast cancer patients exhibiting a short interval (<5 years) to distant metastases from those remaining free of metastases after 5 years were identified. The data set included 78 patients: 44 patients with “good prognosis” (continued to be metastasis-free after at least 5 years) and 34 patients with “poor prognosis” (developed distant metastasis within 5 years). All patients were lymph node negative and under 55 years of age at diagnosis. Gene expression data for each patient was obtained from DNA microarrays containing 24,481 human genes and included the following fields: intensities, intensity ratios, and measurement noise characteristics (P-values).

Veer et.al used a correlation algorithmic approach, based on G-P (Gene-Prognosis) correlation for the supervised data mining procedures. First, G-P correlations were calculated and used to identify the most predictive genes. To identify reliably good and poor prognostic tumors, a three-step supervised classification method was used (see Gruvberger et.al (2001), Cancer Res, 61:5979-5984; Khan et. al (2001) Nature Med., 7:673-679; He et. al, (2001) Nature Med. 7:658-659). Approximately 5,000 genes (significantly regulated in more than 3 tumors out of 78) were selected from the 25,000 genes on the microarray. The correlation coefficient for each gene with disease outcome was calculated and 231 genes were found to be significantly associated with disease outcome (correlation coefficient <−0.3 or >0.3). Second, for each class (good or poor prognosis) in the training data set, a template was derived, representing an average of all expressions of the subset of 231 predictive genes. This template was then used to predict the class (good or poor prognosis) of a “new” patient, by assigning the patient to the class (good or poor) with which its gene expression profile correlated most closely. When this G-P correlation method was tested against the same dataset on which it was trained (all 78 patients) its predictive accuracy was 83% (65 correct predictions out of 78). In a leave-one-out cross-validation test (the prognosis of each patient was predicted by training the algorithm on the other 77 patients), the predictive accuracy dropped to 73%.

The G-P correlation method used in this study to predict disease outcome from gene expression data exhibits two significant weaknesses. First, the gene expression measurement noise is treated inadequately, by simply weighting the data by 1/σ where σ is the standard deviation of the measurement error. Second, the G-P correlation method does not account for G-G (Gene-Gene) correlations, which turned out to be significant in this case (as large as 0.5-0.8 for many pairs). In addition, the use of just two prognosis classes obscures the fact that time to distant metastasis is a continuous variable and a “hard” boundary at 5 years does not take into account the “transition” expression pattern.

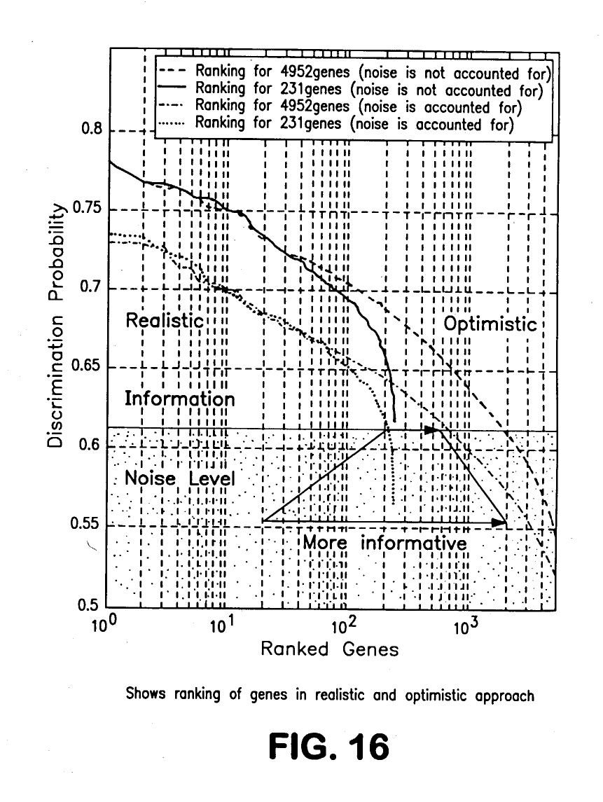

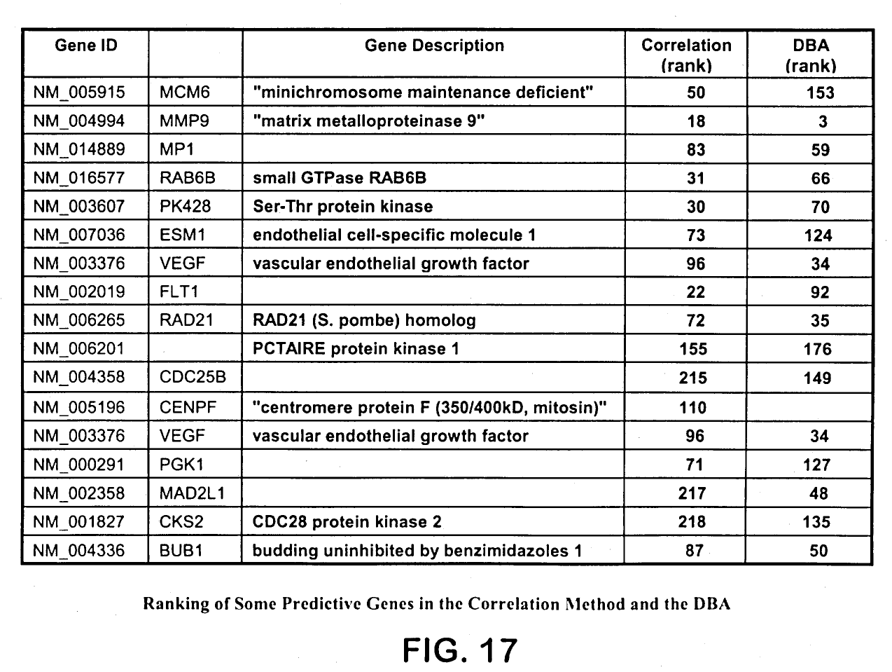

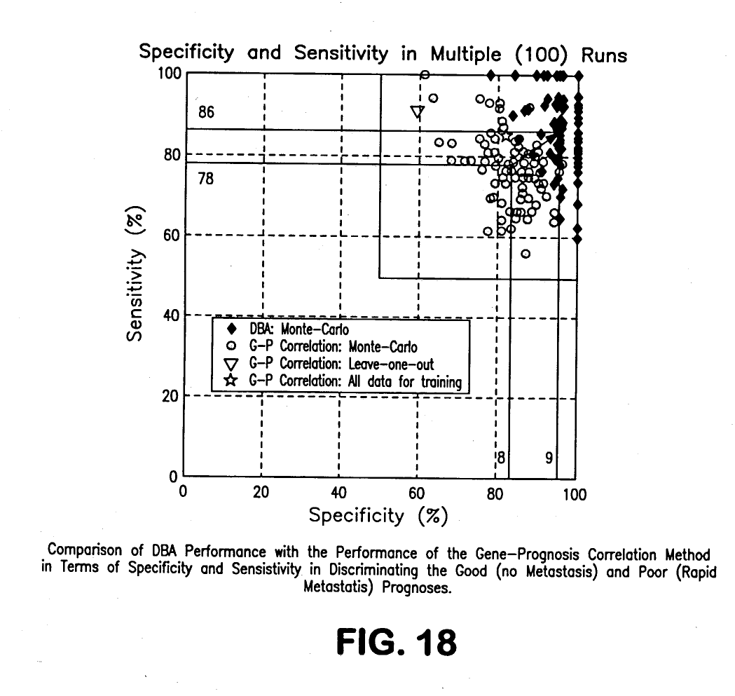

In the DBA, probabilistic approach all significant ˜5000 genes were ranked. FIG. 16 shows that the set of informative genes is expanded to ˜600 genes which carry information beyond the noise level of 0.61 (noise+finite samples) by probabilistic ranking used in the DBA. As shown in FIG. 16, from 231 reporter genes used by Veer et. al, only ˜200 genes have a good probabilistic discrimination. The DBA technology overcomes the weaknesses identified in the Gene-Prognosis correlation method via a rigorous statistical and probabilistic data fusion solution to the full multidimensional nature of gene expression data. First, the DBA adequately treats the gene expression measurement noise by the use of associated uncertainty ellipsoids that sample the full possible space of gene expressions. Second, the DBA accounts for G-G correlations, and uncertainties in their estimates (due to finite samples). Third, the DBA uses an effective global-local estimation of the conditional probability density function for G-P relations, which is a more robust alternative than classification based on linear G-P correlation templates. In particular, this third feature provides for an “implicit” recognition/accounting of transition state patients. FIGS. 17 shows ranking of some predictive genes that are involved in cell cycle; invasion and metastatis; angiogenesis and signal transduction. in the correlation method and the DBA.

To demonstrate the predictive performance of the DBA, intensive Monte-Carlo tests were conducted by randomly dividing the data into multiple training and testing sets. This is the most reliable and realistic cross-validation scheme since it samples G-P relations in the n-dimensional space of genes. FIG. 18 demonstrates that the DBA significantly improves discrimination between the two classes (good and poor prognoses) in realistic Monte-Carlo validation. For example, the DBA (fully accounting for noise and G-G correlations) yields a mean sensitivity of 86% and a mean specificity of 96%, which translate to a mean probability of correct prediction of 92% as shown in Table 1. Specificity and sensitivity results are also shown in FIG. 18 for the G-P correlation method of Veer et. al. In addition to the two tests performed by Veer et al. (apply to same data as trained on, and leave-one-out), FIG. 18 also shows G-P correlation method results in Monte Carlo cross validation scheme. Corresponding probabilities of correct prediction are shown in Table 1.

TABLE 1
Probabilities of Correct Prediction for Different Methods

| | | | | Probability of |
|---|---|---|---|---|
| Feature/ | Cross- | Treatment of | Gene—Gene | correct |
| Method | Validation | Noise | Correlations | prediction (%) |
| DBA | Monte-Carlo | Stastical | Yes | 92 |
| G-P | Monte-Carlo | Weighing | No | 81 |
| Correlation | | data by 1/σ | | |
| G-P | Leave-one- | Weighing | No | 73 |
| Correlation | out | data by 1/σ | | |
| G-P | No (trained | Weighing | No | 83 |
| Correlation | on all data) | data by 1/σ | | |

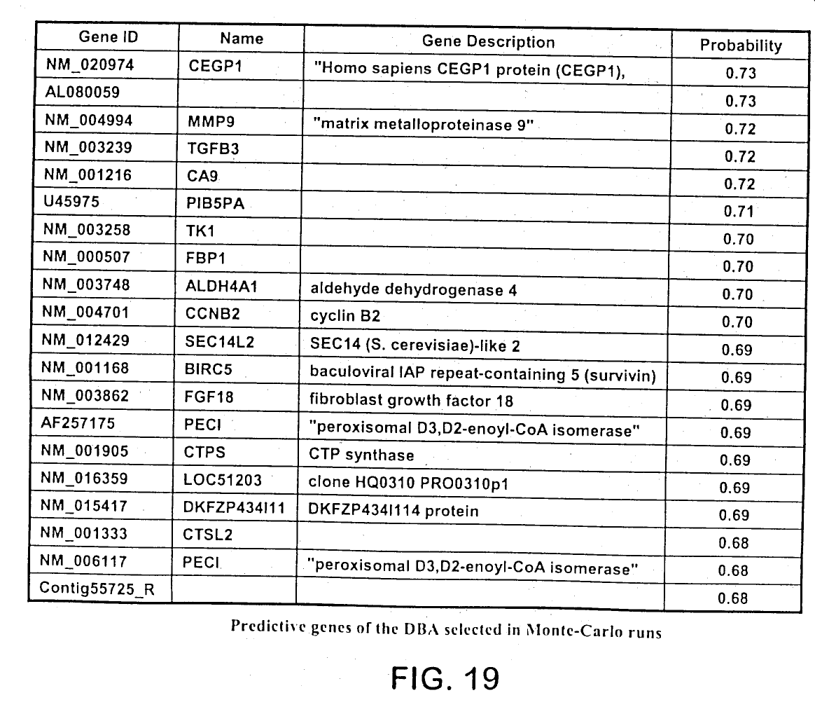

The comparison between the DBA-Monte Carlo and the G-P Correlation-Monte Carlo is shown in FIG. 18 and Table 1. In Table 1, the DBA's probability of correct prediction is 92% compared to 81% for G-P Correlation Monte-Carlo. The DBA when trained on all 78 patients, and then applied it to the same 78 patients, as reported by Veer et. al (83% predictive accuracy), yields predictive results in the 99% range. The use of a sophisticated global-local conditional probability density formulation in DBA, when applied to the same data set it is trained on, result in such high predictability. FIG. 19 shows probabilities of correct prediction for some predictive genes of the DBA selected in Monte-Carlo runs.

Since modifications will be apparent to those of skill in this art, it is intended that this invention be limited only by the scope of the appended claims.

We claim:

1. A method of determining clinically relevant information from gene expression data, comprising:
conducting a statistical analysis of the gene expression data to identify trends and dependencies among the data; and
deriving a probabilistic model from the gene expression data, the probabilistic model being indicative of a probable classification of the data into clinically relevant information.

2. The method of claim 1, wherein the probabilistic model is derived using a discrete Bayesian analysis.

3. The method of claim 1, further comprising generating an empirical probability distribution function (pdf) of stochastic variables in accordance with the gene expression data and extracting information regarding class membership of clinically relevant information with respect to a new set of data values.

4. The method of claim 1 wherein, the gene expression data is processed by:
determining an estimate for one or more hypothesis-conditional probability density functions p(x|Hₖ) for a set X of the gene expression data conditioned on a set H of hypotheses relating to the gene expression data;
determining a set of prior probability density functions p(Hₖ) for each hypothesis of the set H; and
determining a set of posterior test-conditional probability density functions p(Hₖ|x) for the hypotheses conditioned on a new data x;
wherein the p(x|Hᵢ) estimates include a global estimate produced in accordance with uncertainties in the statistical characteristics of the gene expression data relating to each hypothesis-conditional pdf p(x|Hₖ).

5. The method of claim 4, wherein the uncertainties in the statistical characteristics are specified as an ellipsoid about the gene expression data for each hypothesis and each ellipsoid is defined by an m-dimensional ellipsoid E_{q,k} for each hypothesis Hₖ and is specified by:
$${E}_{q , k} = \{ x : {( x - {m}_{x , k} )}^{T} {P}_{x , k}^{- 1} ( x - {m}_{x , k} ) \leq {μ}_{q , k}^{2} \}$$
where the m×1 vector x is the argument in the space of gene expression data, the m×1 vector m_{x,k} is the mean (center) of each ellipsoid, the m×m matrix P_{x,k} is a covariance matrix of the ellipsoid, and the scalar μ²_{q,k} defines the size of the q-th ellipsoid, such that the global estimate of the hypothesis-conditional pdf is specified by:
\hat{P}_{glob}(x/Hₖ)=α_{q,k} if xεE_{q,k}∩E_{q-1,k}(E_{0,k}=E_{1,k}), k=1, . . . , N
for a selected confidence interval parameter α_{q,k}.

6. The method of claim 4, wherein the hypothesis-conditional p(x|Hₖ) estimates further include a local estimate produced in accordance with a discrete neighbor counting process for the gene expression data relative to the global estimate for the corresponding hypothesis-conditional pdf.

7. The method of claim 6, wherein the local estimate for the hypothesis is specified as a probability that an observed vector of tests x and an associated discrete neighbor counting pattern {C_{1,k}(x)},l=1, . . . , Lₖ, k=1, . . . , N might actually be observed, wherein the neighbor counting pattern comprises counting neighbors in the distance layers for each class: {C_{1,k}}, l=1, . . . , Lₖ, wherein the integer C_{1,k} is the number of neighbors associated with the k-th hypothesis whose test values are distanced from a next test value within the 1-th globally-transformed distance layer for the k-th class:
$${C}_{l , k} = {\sum }_{i}^{{n}_{k}} {ϑ}_{l , i , k} , {ϑ}_{l , i , k} = \{ \begin{matrix}1 & if {\overline{d}}_{l - 1 , k} < {d}_{i , k} \leq {\overline{d}}_{l , k} , {\overline{d}}_{0 , k} = 0 \\ 0 & otherwise \end{matrix}$$
where nₖ is the total number of data records in a selected k-th class and the index i runs over all these data records.

8. The method of claim 7, wherein the selected k-th class of the gene expression data corresponds to a selected training subset class of the gene expression data.

9. The method of claim 4, further including:
performing a training mode in which a training subset class of the gene expression data is used to produce the hypothesis-conditional probability density functions p(x|Hₖ); and
performing a prediction mode in which a set of posterior probabilities is determined for the set H of hypotheses, wherein the hypothesis-conditional probability density functions p(x|Hₖ) are produced from the global estimates and from local estimates produced in accordance with a discrete neighbor counting process for the gene expression data relative to the global estimate for the corresponding hypothesis-conditional pdf.

10. The method of claim 9, wherein the local estimate for a hypothesis is specified as a probability that an observed vector of tests x and an associated discrete neighbor counting pattern {C_{1,k}(x)}, l=1, . . . , Lₖ, k=1, . . . , N might actually be observed, wherein the neighbor counting pattern comprises counting neighbors in the distance layers for each class: {C_{1,k}}l=1, . . . , Lₖ, wherein the integer C_{1,k} is the number of test elements associated with the
k-th hypothesis whose test values are distanced from a next test value within the l-th globally-transformed distance layer for the k-th class:
$${C}_{l , k} = {\sum }_{i}^{{n}_{k}} {ϑ}_{l , i , k} , {ϑ}_{l , i , k} = \{ \begin{matrix}1 & if {\overline{d}}_{l - 1 , k} < {d}_{i , k} \leq {\overline{d}}_{l , k} , {\overline{d}}_{0 , k} = 0 \\ 0 & otherwise \end{matrix}$$
where nₖ is the total number of data records in a selected k-th class and the index i runs over all these data records.

11. The method of claim 10, wherein the selected k-th class of the gene expression data corresponds to the training subset class of the gene expression data.

12. The method of claim 1, wherein the posterior test-condition probabilities provide the clinically relevant information.

13. The method of claim 1, wherein the clinically relevant information is compound toxicity; disease diagnosis; disease stage; disease outcome; drug efficacy; or survivability in clinical trials.

14. The method of claim 13, wherein the clinically relevant information is disease diagnosis.

15. The method of claim 14, wherein the diseases are selected from cancer; cardiovascular diseases; diabetes; HIV/AIDS; hepatitis; neurodegenerative diseases; ophthalmic diseases;-blood diseases; respiratory diseases; endocrine diseases; bacterial, fungal parasitic or viral infections; inflammatory diseases; reproductive diseases and any other disease or disorder for which gene expression data can be used to predict clinically relevant information.

16. The method of claim 15, wherein the disease is a cancer, selected from ovarian, lung, pancreatic, prostate, brain, breast, and colon cancer.

17. The method of claim 16, wherein the disease is breast cancer.

18. The method of claim 15, wherein the disease is a cardiovascular disease.

19. The method of claim 18, wherein the cardiovascular disease is selected from arteriosclerosis, angina, high blood pressure, high cholesterol, heart attack, stroke and arrhythmia.

20. The method of claim 15, wherein the disease is a inflammatory disease.

21. The method of claim 20, wherein the inflammatory disease is selected from asthma, chronic obstructive pulmonary disease, rheumatoid arthritis, inflammatory bowel disease, glomerulonephritis, neuroinflammatory disease, multiple sclerosis, and disorders of the immune system.

22. The method of claim 15, wherein the disease is a neurodegenerative disease.

23. The method of claim 19, wherein the neurodegenerative disease is selected from Alzheimer's disease (AD), Parkinson's disease, Huntington's disease, amyotrophic lateral sclerosis (ALS), and other brain disorders.

24. The method of claim 15, wherein the disease is a pulmonary disease.

25. The method of claim 1, wherein the gene expression data is obtained from the levels of genes expressed, particular genes expressed, post-translational modifications of genes, encoded proteins that are expressed, glycosylation or splice variants of genes.

26. The method of claim 1, further comprising an update step in which new data is convolved with the a priori probability of a discretized state vector of a hypothesis to generate the a posteriori probability of the hypothesis.

27. The method of claim 26, further comprising a prediction step wherein trends in the data are captured via Markov chain models of the discretized state.

28. The method of claim 1, further comprising compiling data into a database.

29. A method for generating an a posteriori tree of clinically relevant information for a subject, wherein the method comprises:
performing an analysis of gene expression data for a population of individuals, wherein the data comprises a matrix of discriminations between clinically relevant information selected from a predetermined list of clinically relevant information;
performing a Bayesian statistical analysis to estimate a series of hypothesis-conditional probability density functions p(x|Hi) where a hypothesis Hi is one of a set H of the clinically relevant information;
determining a prior probability density function p(Hi) for each of the hypotheses Hi;
determining a posterior test-conditional probability density function p(Hi|x) for each of the hypotheses Hi test data records; and
generating a posterior tree of possible clinically relevant information for a test subject in accordance with test results for the test subject.

30. The method of claim 29, wherein the matrix of discriminations is a pair-wise matrx.

31. A method of developing a test to screen for one or more inapparent diseases, comprising:
conducting a statistical analysis of data in order to identify trends and dependencies among the data, wherein the data comprises gene expression data from a subject;
deriving a probabilistic model from the data, the probabilistic model being indicative of a probable disease diagnosis for a patient, wherein the probabilistic model is derived using a discrete Bayesian analysis;
identifying from among the input data, the data that contributes to the diagnosis; and
identifying the genes that generated the data that contributes to the diagnosis.

32. A method of diagnosing a disease condition of a patient, the method comprising:
receiving a set of gene expression data comprising gene expression data from a population X of individuals;
estimating a hypothesis-conditional probability density function p(x|H 1) where the hypothesis H1 relates to a diagnosis condition for a test patient x, and estimating a hypothesis-conditional probability density function p(x|H2) where the hypothesis H2 relates to a non-diagnosis condition for a test patient;
determining a prior probability density function p(H) for the each of the hypotheses H1 and H2;
determining a posterior test-conditional probability density function p(H|x) for each of the hypotheses H1 and H2 on the test data x; and
providing a diagnosis probability of a new patient for the H disease condition, based on the determined posterior test-conditional probability density function p(H1|x) as compared to the posterior test-conditional probability density function p(H2|x) and gene expression data of the new patient.

33. The method of claim 32, wherein the diseases are selected from cancer; cardiovascular diseases; diabetes; HIV/AIDS; hepatitis; neurodegenerative diseases; ophthalmic diseases; blood diseases; respiratory diseases; endocrine diseases; bacterial, fungal parasitic or viral infections; inflammatory diseases; reproductive diseases and any other disease or disorder for which gene expression data can be used to predict clinically relevant information.

34. A method of diagnosing a disease from data, comprising:
conducting a statistical analysis of the data in order to identify trends and dependencies among the data, wherein the data comprises gene expression data from a subject;
deriving a probabilistic model from the data, the probabilistic model being indicative of a probable disease diagnosis for a patient, wherein the disease is an inapparent disease.

35. The method of claim 34, wherein the probabilistic model is derived using a discrete Bayesian analysis.

36. The method of claim 34, further comprising compiling data into a database.

37. The method of claim 34, further comprising an update step in which new data is convolved with the a priori probability of a discretized state vector of a hypothesis to generate the a posteriori probability of the hypothesis.

38. The method of claim 36, further comprising a prediction step wherein trends in the data are captured via Markov chain models of the discretized state.
